SEQUENCE LISTING (82 sequences)

SEQ ID NO: 1 - protein, 444 aa, synthetic construct
MILSWKRLRGGRGRRAGKKQPGVESGGEMELTTPNHFRCPISLDLMKDPVTLSTGITYDRENIEKWIHEDGNITCPVTNRVLTSFEPIPNHTIRRMIQDWCVENRSYGIERIPTPRIPVSSVEVFEINCNITTACKSEDQAGCRDLVAKIKKWTKESDRNKRCIVDNGAVSVLAEAFETFSKTCLDENVSVLEEILSTLTLLFPLAGEGLTYLGSASSMRCMVWFLKSGDLSRRRNAVLVLREIVSSDHRKVNVLLDIEGAIEPLFKLIKEPICPTATKASLVVVYRTITSASATEKPIQKFVDMGLVSLLLEMLVDAQRSLCEKALSVLDGLCSSDYGRGDAYNNSLIFPVIVKKILRVSDLATEFAVSILWKLCKNEEREEKTAFAEALQVGAFQKLLLLLQVGCADKTKEHVSELLKLLNPHRARLECIDSMDFKDLKRPF

SEQ ID NO: 2 - DNA, 1335 bp, synthetic construct
  source 1..1335: mol_type = other DNA
atgattttgtcatggaaaagactaagaggcggccggggccggcgtgccggcaagaagcagccgggggtagaaagcgggggagagatggagttgacgacgccgaaccacttccggtgcccgatatctctcgacttgatgaaagatccggtgactttatccactgggattacgtatgatcgagagaatatcgagaagtggattcatgaggatggaaacatcacctgccccgttacaaaccgagtcctgacgagttttgaacccattccgaatcacacaatccgtcggatgatacaagattggtgtgtcgagaaccggtcttacggcatcgagagaatcccgacgcccagaattcctgtgagttcggttgaggttttcgagatcaattgtaatatcacgacggcttgcaaaagtgaagatcaagctgggtgcagagatttggtggcaaagatcaagaaatggaccaaggagagtgaccggaacaagaggtgcattgtggacaacggagctgtaagtgttttggcggaagcgtttgaaacattttcgaaaacgtgtttggatgaaaatgtttctgttttggaggaaatactgtcgactttgacgttgctgtttcctcttgcgggggaagggttgacttacttgggatcggcttcgtccatgcgttgcatggtctggttcttgaagagcggagatttgtcaaggaggagaaacgccgtgttggtgttgagagagatcgtttcttcggatcatagaaaagttaacgtgttgttggatattgaaggagccattgaaccgctatttaagctgatcaaagagcccatttgccccactgctactaaagcttcgttggtcgtcgtctatcgtacgatcacatcagcttctgcgactgagaaaccaatacaaaagtttgtcgacatggggttggtgtcattgctgctcgaaatgcttgtcgatgctcaacgaagcctatgcgagaaggcgttgagtgttctcgacggactttgcagcagtgattatgggagaggagatgcctacaataattctttaatattcccggttattgtgaagaaaatactgcgagtttcagatttggcgacggagttcgcagtctccattctgtggaagctatgcaagaatgaagaaagagaagagaaaactgcttttgccgaagcgcttcaagtgggtgcttttcagaagctgttgttgcttttgcaagttggttgtgctgacaagacgaaggagcatgtgtctgagttgctgaagctgttgaatcctcatagagctagattggaatgcattgattccatggatttcaaggacctgaaaaggccgttttga

SEQ ID NO: 3 - DNA, 606 bp, synthetic construct
  source 1..606: mol_type = other DNA
atgattttgtcatggaaaagactaagaggcggccggggccggcgtgccggcaagaagcagccgggggtagaaagcgggggagagatggagttgacgacgccgaaccacttccggtgcccgatatctctcgacttgatgaaagatccggtgactttatccactgggattacgtatgatcgagagaatatcgagaagtggattcatgaggatggaaacatcacctgccccgttacaaaccgagtcctgacgagttttgaacccattccgaatcacacaatccgtcggatgatacaagattggtgtgtcgagaaccggtcttacggcatcgagagaatcccgacgcccagaattcctgtgagttcggttgaggttttcgagatcaattgtaatatcacgacggcttgcaaaagtgaagatcaagctgggtgcagagatttggtggcaaagatcaagaaatggaccaaggagagtgaccggaacaagaggtgcattgtggacaacggagctgtaagtgttttggcggaagcgtttgaaacattttcgaaaacgtgtttggatgaaaatgtttctgttttggaggaaatactgtcgactttgacgttgctg

SEQ ID NO: 4 - DNA, 800 bp, synthetic construct
  source 1..800: mol_type = other DNA
atgattttgtcatggaaaagactaagaggcggccggggccggcgtgccggcaagaagcagccgggggtagaaagcgggggagagatggagttgacgacgccgaaccacttccggtgcccgatatctctcgacttgatgaaagatccggtgactttatccactgggattacgtatgatcgagagaatatcgagaagtggattcatgaggatggaaacatcacctgccccgttacaaaccgagtcctgacgagttttgaacccattccgaatcacacaatccgtcggatgatacaagattggtgtgtcgagaaccggtcttacggcatcgagagaatcccgacgcccagaattcctgtgagttcggttgaggttttcgagatcaattgtaatatcacgacggcttgcaaaagtgaagatcaagctgggtgcagagatttggtggcaaagatcaagaaatggaccaaggagagtgaccggaacaagaggtgcattgtggacaacggagctgtaagtgttttggcggaagcgtttgaaacattttcgaaaacgtgtttggatgaaaatgtttctgttttggaggaaatactgtcgactttgacgttgctgtttcctcttgcgggggaagggttgacttacttgggatcggcttcgtccatgcgttgcatggtctggttcttgaagagcggagatttgtcaaggaggagaaacgccgtgttggtgttgagagagatcgtttcttcggatcatagaaaagttaacgtgttgttggatattgaaggagccattgaaccgctatttaa

SEQ ID NO: 5 - protein, 444 aa, synthetic construct
MILSWKRLRGGRGRRAGKKQPGVESGGEMELTTPNHFRAPISLDLMKDPVTLSTGITYDRENIEKWIHEDGNITCPVTNRVLTSFEPIPNHTIRRMIQDWCVENRSYGIERIPTPRIPVSSVEVFEINCNITTACKSEDQAGCRDLVAKIKKWTKESDRNKRCIVDNGAVSVLAEAFETFSKTCLDENVSVLEEILSTLTLLFPLAGEGLTYLGSASSMRCMVWFLKSGDLSRRRNAVLVLREIVSSDHRKVNVLLDIEGAIEPLFKLIKEPICPTATKASLVVVYRTITSASATEKPIQKFVDMGLVSLLLEMLVDAQRSLCEKALSVLDGLCSSDYGRGDAYNNSLIFPVIVKKILRVSDLATEFAVSILWKLCKNEEREEKTAFAEALQVGAFQKLLLLLQVGCADKTKEHVSELLKLLNPHRARLECIDSMDFKDLKRPF

SEQ ID NO: 6 - DNA, 1335 bp, synthetic construct
  source 1..1335: mol_type = other DNA
atgattttgtcatggaaaagactaagaggcggccggggccggcgtgccggcaagaagcagccgggggtagaaagcgggggagagatggagttgacgacgccgaaccacttccgggccccgatatctctcgacttgatgaaagatccggtgactttatccactgggattacgtatgatcgagagaatatcgagaagtggattcatgaggatggaaacatcacctgccccgttacaaaccgagtcctgacgagttttgaacccattccgaatcacacaatccgtcggatgatacaagattggtgtgtcgagaaccggtcttacggcatcgagagaatcccgacgcccagaattcctgtgagttcggttgaggttttcgagatcaattgtaatatcacgacggcttgcaaaagtgaagatcaagctgggtgcagagatttggtggcaaagatcaagaaatggaccaaggagagtgaccggaacaagaggtgcattgtggacaacggagctgtaagtgttttggcggaagcgtttgaaacattttcgaaaacgtgtttggatgaaaatgtttctgttttggaggaaatactgtcgactttgacgttgctgtttcctcttgcgggggaagggttgacttacttgggatcggcttcgtccatgcgttgcatggtctggttcttgaagagcggagatttgtcaaggaggagaaacgccgtgttggtgttgagagagatcgtttcttcggatcatagaaaagttaacgtgttgttggatattgaaggagccattgaaccgctatttaagctgatcaaagagcccatttgccccactgctactaaagcttcgttggtcgtcgtctatcgtacgatcacatcagcttctgcgactgagaaaccaatacaaaagtttgtcgacatggggttggtgtcattgctgctcgaaatgcttgtcgatgctcaacgaagcctatgcgagaaggcgttgagtgttctcgacggactttgcagcagtgattatgggagaggagatgcctacaataattctttaatattcccggttattgtgaagaaaatactgcgagtttcagatttggcgacggagttcgcagtctccattctgtggaagctatgcaagaatgaagaaagagaagagaaaactgcttttgccgaagcgcttcaagtgggtgcttttcagaagctgttgttgcttttgcaagttggttgtgctgacaagacgaaggagcatgtgtctgagttgctgaagctgttgaatcctcatagagctagattggaatgcattgattccatggatttcaaggacctgaaaaggccgttttga

SEQ ID NO: 7 - protein, 519 aa, synthetic construct
MKDMSIPLLAAVSSSTEETVRPIADFHPTLWGNHFLKSAADVETIDAATQEQHAALKQEVRRMITTTANKLAQKLHMIDAVQRLGVAYHFEKEIEDELGKVSHDLDSDDLYVVSLRFRLFRQQGVKISCDVFDKFKDDEGKFKESLINDIRGMLSLYEAAYLAIRGEDILDEAIVFTTTHLKSVISISDHSHVNSNLAEQIRHSLQIPLRKAAARLEARYFLDIYSRDDLHDETLLKFAKLDFNILQAAHQKEASIMTRTKDISEVDTILGVKVKKHSGERANISIIFDFFRWDIGLIDTLPEYMKFIVKALLDIYREAEEELAKEGRSYGIPYAKQMMQELIILYFTEAKWLYKGYVPTFDEYKSVALRSIGLRTLAVASFVDLGDFIATKDNFECILKNAKSLKATETIGRLMDDIAGYKFEQKRGHNPSAVECYKNQHGVSEEEAVKELLLEVANSWKDINEELLNPTTVPLPMLQRLLYFARSGHFIYDDGHDRYTHSLMMKRQVALLLTEPLAI

SEQ ID NO: 8 - DNA, 1560 bp, synthetic construct
  source 1..1560: mol_type = other DNA
atgaaagatatgtctattccacttctagcagcagtttccagttcaactgaagaaactgttcgtcccattgcagattttcatcctactttatggggaaaccatttcctgaaatccgctgccgatgtcgagacaattgacgctgcaactcaagaacagcacgcagcactgaagcaagaggtcaggaggatgataaccactactgccaataagcttgcccaaaaactacacatgattgatgcagtacaacgattaggtgtcgcttatcattttgaaaaagagattgaagatgaattgggaaaggtatctcatgatcttgacagtgatgatctatacgttgtttctcttcgttttcgactttttagacagcaaggagttaagatttcatgtgatgtgtttgataagttcaaagatgatgaaggtaaattcaaggaatcattgatcaacgatatacgaggcatgttgagtttgtacgaggcagcatacctagcaattcggggggaagacattttagatgaagccattgttttcactaccactcaccttaagtcagtaatatctatatctgatcattctcatgtaaactctaatcttgctgaacaaatacgtcattctctgcaaattcctctccgtaaagccgcagcaaggttagaggcaaggtattttttggatatctattcaagggatgatttgcatgatgaaactttgctcaagtttgcaaagttagactttaatatattacaagcagcacaccagaaggaagcaagtatcatgaccagaacgaaagatattagtgaagtagatactatcttaggtgtcaaagtgaaaaaacatagtggggaacgtgcaaatatatcgattatatttgattttttcagatgggacattggcctcatagatacactgccagaatacatgaaatttattgtcaaggcactcttagatatttatagagaagctgaggaagaattggcaaaggaaggaagatcgtacggcataccatatgcaaaacaaatgatgcaagaattaattattttgtactttaccgaagccaagtggttatataaaggttatgttccaaccttcgatgaatacaaatcagttgcattaagaagtatcggtttacgtacgctagcggtcgcttccttcgttgatttgggtgattttattgcaacgaaggacaattttgaatgcattcttaaaaacgctaagagtcttaaagcgacagaaaccattggcaggctcatggatgacatagcaggttataagtttgagcagaagagaggacataatccctcagcagttgagtgctacaagaatcaacatggcgtctccgaggaagaggcagttaaagagttgttgttagaagttgctaacagttggaaagatataaatgaggagcttctcaacccaacgactgtcccgttacctatgcttcaacggcttctttatttcgcacgttcaggccattttatctacgacgatggtcatgatcgctacacccattcattgatgatgaaacgccaagttgccttgttgctgacggaaccccttgcgatctga

SEQ ID NO: 9 - DNA, 33 bp, synthetic construct
  source 1..33: mol_type = other DNA
caagggtaccatgattttgtcatggaaaagact

SEQ ID NO: 10 - DNA, 30 bp, synthetic construct
  source 1..30: mol_type = other DNA
caagctcgagaacggccttttcaggtcctt

SEQ ID NO: 11 - DNA, 30 bp, synthetic construct
  source 1..30: mol_type = other DNA
caagctcgagcagcaacgtcaaagtcgaca

SEQ ID NO: 12 - DNA, 30 bp, synthetic construct
  source 1..30: mol_type = other DNA
caagctcgagttaaatagcggttcaatggc

SEQ ID NO: 13 - DNA, 24 bp, synthetic construct
  source 1..24: mol_type = other DNA
tataaaggttgacctttcgagttt

SEQ ID NO: 14 - DNA, 25 bp, synthetic construct
  source 1..25: mol_type = other DNA
acaaaagcagaaatagcacgaacaa

SEQ ID NO: 15 - DNA, 20 bp, synthetic construct
  source 1..20: mol_type = other DNA
cgttggtcgtcgtctatcgt

SEQ ID NO: 16 - DNA, 20 bp, synthetic construct
  source 1..20: mol_type = other DNA
aatggagactgcgaactccg

SEQ ID NO: 17 - DNA, 22 bp, synthetic construct
  source 1..22: mol_type = other DNA
gtatgccacgtcgcattccaga

SEQ ID NO: 18 - DNA, 22 bp, synthetic construct
  source 1..22: mol_type = other DNA
gccaaaactgctaagggcattc

SEQ ID NO: 19 - DNA, 21 bp, synthetic construct
  source 1..21: mol_type = other DNA
gtcgagcgcgtatgcaatacg

SEQ ID NO: 20 - DNA, 24 bp, synthetic construct
  source 1..24: mol_type = other DNA
gcgttatcccgtagaaaaaggtag

SEQ ID NO: 21 - DNA, 23 bp, synthetic construct
  source 1..23: mol_type = other DNA
ggtatgccacgtcgcattccaga

SEQ ID NO: 22 - DNA, 22 bp, synthetic construct
  source 1..22: mol_type = other DNA
gccaaaactgctaagggcattc

SEQ ID NO: 23 - DNA, 32 bp, synthetic construct
  source 1..32: mol_type = other DNA
caagggatccatgattttgtcatggaaaagac

SEQ ID NO: 24 - DNA, 30 bp, synthetic construct
  source 1..30: mol_type = other DNA
caagaggcctaaacggccttttcaggtcct

SEQ ID NO: 25 - DNA, 33 bp, synthetic construct
  source 1..33: mol_type = other DNA
ccgaaccacttccgggccccgatatctctcgac

SEQ ID NO: 26 - DNA, 30 bp, synthetic construct
  source 1..30: mol_type = other DNA
tctttcatcaagtcgagagatatcggggcc

SEQ ID NO: 27 - DNA, 20 bp, synthetic construct
  source 1..20: mol_type = other DNA
ccgggggtagaaagcggggg

SEQ ID NO: 28 - DNA, 20 bp, synthetic construct
  source 1..20: mol_type = other DNA
acaattgatctcgaaaacct

SEQ ID NO: 29 - DNA, 20 bp, synthetic construct
  source 1..20: mol_type = other DNA
tcgggggaagaaagcgggag

SEQ ID NO: 30 - DNA, 20 bp, synthetic construct
  source 1..20: mol_type = other DNA
agaattgatttccaaaacct

SEQ ID NO: 31 - DNA, 40 bp, synthetic construct
  source 1..40: mol_type = other DNA
cgacggccagtgccaagcttgcgctcaggagccggttgaa

SEQ ID NO: 32 - DNA, 31 bp, synthetic construct
  source 1..31: mol_type = other DNA
gtgttggtctcgcaaccaacctgggagtcct

SEQ ID NO: 33 - DNA, 33 bp, synthetic construct
  source 1..33: mol_type = other DNA
actgcttgctgcttggcagacatactgtcccac

SEQ ID NO: 34 - DNA, 33 bp, synthetic construct
  source 1..33: mol_type = other DNA
tcctagggaagcttagctcttacctgttttcgt

SEQ ID NO: 35 - DNA, 665 bp, synthetic construct
  source 1..665: mol_type = other DNA
gcgctcaggagccggttgaatttgattgttgtttgatgtttaggtatgcttacaattttttactaaataaggtaataattgttccttttattttattcatttagcttggtacttgtcgtaatctatctccgtcaatgtcagcttcttctgtgtcggaagaagctgaaaaatgttgttgaaggccctttcatttattattatcattattatgtttaattttgttcggcaaatgtgtactaatttggttcgaatcgttcgatctcataatacaaacatgtaatgagttatttatgacgtcatggatgcggttagtttgcgtggtctgaaatttcaaccaaattattcaatcatggtggtggccggctggtgcctatcccttgattgaaatatgcaattaaaattctcgtaataatgttgatttgttctatatacttgaattgatggtattaattattgttataaaagcactaacttgtttgagaaaggggataactaaaaggtaataataataataataaaataaagaaaaggcccaacacatgggcgccctatctgttgggccaaatgccataagaggatccagctagcccgttgaagaaaatcccacatcgaaagaaaaaactgaataaacatggtctatatatacaaggactcccaggttggtt

SEQ ID NO: 36 - DNA, 465 bp, synthetic construct
  source 1..465: mol_type = other DNA
tggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagctt

SEQ ID NO: 37 - DNA, 31 bp, synthetic construct
  source 1..31: mol_type = other DNA
tcccaggttggttgcgagaccaacacaaggt

SEQ ID NO: 38 - DNA, 32 bp, synthetic construct
  source 1..32: mol_type = other DNA
gtatgtctgccaagcagcaagcagtatcgatc

SEQ ID NO: 39 - DNA, 33 bp, synthetic construct
  source 1..33: mol_type = other DNA
aagagctaagcttccctaggatggcccctaaga

SEQ ID NO: 40 - DNA, 34 bp, synthetic construct
  source 1..34: mol_type = other DNA
gatcctccgctcttaagcttcttcttcttcgcct

SEQ ID NO: 41 - DNA, 328 bp, synthetic construct
  source 1..328: mol_type = other DNA
tcccaggttggttgcgagaccaacacaaggtctcacgttttgcattgagttttctccgtcgcatgtttgcagttttattttccgttttgcattgaaatttctccgtctcatgtttgcagcgtgttcaaaaagtacgcagctgtatttcacttatttacggcgccacattttcatgccgtttgtgccaactatcccgagctagtgaatacagcttggcttcacacaacactggtgacccgctgacctgctcgtacctcgtaccgtcgtacggcacagcatttggaattaaagggtgtgatcgatactgcttgctgcttggcagacatac

SEQ ID NO: 42 - DNA, 5206 bp, synthetic construct
  source 1..5206: mol_type = other DNA
aagagctaagcttccctaggatggcccctaagaagaagagaaaggtcggtattcacggcgttcctgcggcgtcaaagtcttggggcaagttcatcgaggaggaggaggccgagatggcgtcaaggcgcaacctcatgattgtcgacggcaccaatctgggcttccggttcaagcacaacaattctaagaagcctttcgcctccagctacgtgtccacaatccagagcctcgccaagtcctacagcgcgcgcaccacaattgtgctgggcgacaagggcaagtcagtcttccggctggagcatctgccggagtacaagggcaacagggatgagaagtacgcacagaggaccgaggaggagaaggcactcgatgagcagttcttcgagtacctcaaggacgccttcgagctgtgcaagaccacattcccaaccttcacaatcaggggagtggaggcagacgatatggcagcgtacatcgtcaagctcattggccacctgtacgatcatgtgtggctcatttccacagacggcgattgggacaccctcctgacagacaaggtttcacggttctctttcaccacacggagggagtaccacctgagggatatgtacgagcaccataacgtggacgatgtcgagcagttcatcagcctcaaggccattatgggcgatctgggcgacaatatcaggggagtcgagggaattggagcaaagaggggctacaacatcattcgggagttcggcaatgtgctcgatatcattgaccagctcccgctgccaggcaagcagaagtacatccagaacctcaatgcgtccgaggagctcctgttccgcaatctcatcctggtggatctgccgacctactgcgtcgacgcaattgcagcagtgggacaggatgtcctcgacaagttcacaaaggatatcctggagattgcggagcagggtggaggcggaagtggaggtggcgggtcagggggtggcggatctcctagggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatgccatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagcttaagagcggaggatc

SEQ ID NO: 43 - DNA, 169 bp, synthetic construct
  source 1..169: mol_type = other DNA
ttgtgcagatgatccgtggcgtgagaccctgcagggtctctgttttagagctagaaatagcaagttaaaataaggctagtccgttatcaacttgaaaaagtggcaccgagtcggtgcaaccacttccgggccccgatatctttttttttcgttttgcattgagttttct

SEQ ID NO: 44 - DNA, 20 bp, synthetic construct
  source 1..20: mol_type = other DNA
tgcaacagtatggcgtgtct

SEQ ID NO: 45 - DNA, 20 bp, synthetic construct
  source 1..20: mol_type = other DNA
ccaccatcatccgagcaaga

SEQ ID NO: 46 - DNA, 20 bp, synthetic construct
  source 1..20: mol_type = other DNA
ttggcgtggcctatcacttt

SEQ ID NO: 47 - DNA, 20 bp, synthetic construct
  source 1..20: mol_type = other DNA
atgttgccgtagaagccgaa

SEQ ID NO: 48 - DNA, 20 bp, synthetic construct
  source 1..20: mol_type = other DNA
gaccttggcagcgatagtga

SEQ ID NO: 49 - DNA, 20 bp, synthetic construct
  source 1..20: mol_type = other DNA
gtgtgctgcctcgtacaaac

SEQ ID NO: 50 - DNA, 20 bp, synthetic construct
  source 1..20: mol_type = other DNA
ggcagcacacctagccatta

SEQ ID NO: 51 - DNA, 20 bp, synthetic construct
  source 1..20: mol_type = other DNA
tagggctttacggagtggga

SEQ ID NO: 52 - DNA, 20 bp, synthetic construct
  source 1..20: mol_type = other DNA
tccttggcatgtgtgaggtt

SEQ ID NO: 53 - DNA, 22 bp, synthetic construct
  source 1..22: mol_type = other DNA
agcattcaactcctgtggtaac

SEQ ID NO: 54 - DNA, 23 bp, synthetic construct
  source 1..23: mol_type = other DNA
tggcaaagaaaagatgcaagagc

SEQ ID NO: 55 - DNA, 25 bp, synthetic construct
  source 1..25: mol_type = other DNA
tgatgacgctgttgaaatcttaggg

SEQ ID NO: 56 - DNA, 20 bp, synthetic construct
  source 1..20: mol_type = other DNA
gaaaccattggcaggctcat

SEQ ID NO: 57 - DNA, 21 bp, synthetic construct
  source 1..21: mol_type = other DNA
cgtgcgaaataaagaagccgt

SEQ ID NO: 58 - DNA, 21 bp, synthetic construct
  source 1..21: mol_type = other DNA
tgacgaatgacgttgaaggca

SEQ ID NO: 59 - DNA, 22 bp, synthetic construct
  source 1..22: mol_type = other DNA
taaccttgatacggctctacgg

SEQ ID NO: 60 - DNA, 22 bp, synthetic construct
  source 1..22: mol_type = other DNA
agtggtttccatgacttctgct

SEQ ID NO: 61 - DNA, 22 bp, synthetic construct
  source 1..22: mol_type = other DNA
aggcagtctaatgcaactgtca

SEQ ID NO: 62 - DNA, 21 bp, synthetic construct
  source 1..21: mol_type = other DNA
gcaaccgtttccagttcaaca

SEQ ID NO: 63 - DNA, 20 bp, synthetic construct
  source 1..20: mol_type = other DNA
attgcaacttctgggcaggt

SEQ ID NO: 64 - DNA, 31 bp, synthetic construct
  source 1..31: mol_type = other DNA
caagggtaccatgaaagatatgtctattcca

SEQ ID NO: 65 - DNA, 30 bp, synthetic construct
  source 1..30: mol_type = other DNA
caaggaattcatcgcaaggggttccgtcag

SEQ ID NO: 66 - DNA, 30 bp, synthetic construct
  source 1..30: mol_type = other DNA
caaggaattcatgacggactaccggttacc

SEQ ID NO: 67 - DNA, 30 bp, synthetic construct
  source 1..30: mol_type = other DNA
caagaggcctttgggtatctccaactttgg

SEQ ID NO: 68 - DNA, 18 bp, synthetic construct
  source 1..18: mol_type = other DNA
cacttccggtgcccgata

SEQ ID NO: 69 - DNA, 18 bp, synthetic construct
  source 1..18: mol_type = other DNA
cacttccgggccccgata

SEQ ID NO: 70 - protein, 38 aa, synthetic construct
APSHFFCPILQEVMEDPYIAADGFTYEHRAIKAWLEKH

SEQ ID NO: 71 - protein, 39 aa, synthetic construct
CPEEFKCPLSKELMRDPVILASGQTFDRPYIQRWLKAGN

SEQ ID NO: 72 - protein, 39 aa, synthetic construct
IPADFLCPISLELMRDPVIVATGQTYERSYIQRWIDCGN

SEQ ID NO: 73 - protein, 39 aa, synthetic construct
VPADFRCPISLELMRNPVVVATGQTYDRQSISLWIESGH

SEQ ID NO: 74 - protein, 40 aa, synthetic construct
TPNHFRCPISLDLMKDPVTLSTGITYDRENIEKWIHEDGN

SEQ ID NO: 75 - protein, 40 aa, synthetic construct
TPNHFRCPISLDLMKDPVTLSTGITYDRENIEKWIHENGN

SEQ ID NO: 76 - protein, 39 aa, synthetic construct
IPPEFQCPISIDLMKDPVIISTGITYDRVSIETWINSGN

SEQ ID NO: 77 - protein, 39 aa, synthetic construct
VPRDFRCPISLDLMKDPVTLSTGITYDRENIEKWIEAGN

SEQ ID NO: 78 - protein, 39 aa, synthetic construct
VPSHFKCPISLDLMSDPVTLSTGITYDRVSIETWIENGN

SEQ ID NO: 79 - protein, 40 aa, synthetic construct
TPNHFRAPISLDLMKDPVTLSTGITYDRENIEKWIHEDGN

SEQ ID NO: 80 - DNA, 31 bp, synthetic construct
  source 1..31: mol_type = other DNA
aaccacttccggtgcccgatatctctcgact

SEQ ID NO: 81 - DNA, 23 bp, synthetic construct
  source 1..23: mol_type = other DNA
ccggtgcccgatatctctcgact

SEQ ID NO: 82 - DNA, 24 bp, synthetic construct
  source 1..24: mol_type = other DNA
aaccacttccgggccccgatatct

(19) United States
(12) Patent Application Publication
(10) Pub. No.: US 2026/0185118 A1
(43) Pub. Date: Jul. 2, 2026

(54) METHOD FOR CULATIVATING HUANGLONGBING-RESISTANT PLANT AND APPLICATION THEREOF

(71) Applicant: INSTITUTE OF MICROBIOLOGY, CHINESE ACADEMY OF SCIENCES, Beijing (CN)

(72) Inventors: Jian Ye, Beijing (CN); Pingzhi Zhao, Beijing (CN); Yanwei Sun, Beijing (CN); Huan Yang, Beijing (CN)

(73) Assignee: INSTITUTE OF MICROBIOLOGY, CHINESE ACADEMY OF SCIENCES, Beijing (CN)

(21) Appl. No.: 19/130,530

(22) PCT Filed: Feb. 8, 2024
(86) PCT No.: PCT/CN2024/076842

§ 371 Date: May 15, 2025

(30) Foreign Application Priority Data
Feb. 9, 2023 (CN) 202310086555.1

(51) Int. Cl. C12N 15/82; C12N 9/00; C12N 15/52

(52) U.S. Cl. CPC C12N 15/8286; C12N 9/93; C12N 15/52; C12N 15/8202; C12N 15/8281; C12Y 603/02019

(57) ABSTRACT

A method and application for cultivating a Huanglongbing-resistant plant is provided. CsPUB21 attenuates a defense response of citrus based on terpene compounds through direct ubiquitination and degradation of a CsMYC2 protein. Resistance of citrus to a pathogen CLas and a spread vector thereof, i.e. Asian Diaphorina citri Kuwayama, can be improved by silencing of a CsPUB21 gene. Infestation of the pathogen CLas can be reduced by overexpression of CsPUB21DN, a dominant negative mutant of PUB21. Additionally, the CLas concentration in leaves of a susceptible plant can be effectively reduced by artificially replacing an amino acid Cys (TGC) at position 39 of a key enzyme activation site of CsPUB21 in the susceptible plant with Ala (GCC) via gene editing. This method and application provide a means to effectively resist the replication and spread of Huanglongbing pathogens, which cause citrus greening.

### CROSS-REFERENCE TO RELATED APPLICATION

The present application is a national phase entry of International Application No. PCT/CN2024/076842 filed on Feb. 8, 2024, which claims benefit to the Chinese Patent Application No. 202310086555.1, filed on Feb. 9, 2023. The entire contents of these applications are incorporated herein by reference.

### SEQUENCE LISTING

This application contains a sequence listing in computer readable form (file name: 24005.xml; date of creation: May 15, 2025; file size 86 kb) which is incorporated herein by reference in its entirety and forms part of the disclosure.

### TECHNICAL FIELD

The present disclosure belongs to the field of biotechnology, and specifically relates to a method for cultivating a Huanglongbing-resistant plant, and application thereof, in particular to a method for cultivating a Huanglongbing-resistant plant based on a PUB21 protein and a mutant thereof, and application thereof.

### BACKGROUND

Citrus huanglongbing (HLB), the most destructive disease for citrus, is caused by a phloem-limited bacterium, Candidatus Liberibacter asiaticus (CLas), and is mainly transmitted by an insect vector, Asian Diaphorina citri Kuwayama.

Recent research reports have demonstrated that citrus Huanglongbing is an immune disease caused by pathogens. Resistance genes (R genes) function as immune systems in plants by directly or indirectly recognizing pathogens and triggering defense pathways, and deployment of the R genes in molecular breeding is the most effective and sustainable way to control immune diseases of crops. Generally, wild species containing R genes are hybridized with cultivated species, resulting in infiltration of the R genes into the cultivated species, which creates resistance to pathogens, but to date, no R gene resistant to Huanglongbing has been found in wild citrus varieties or closely related genera.

An emerging strategy for persistent and broad-spectrum resistance breeding is to destroy susceptibility genes (S genes) of the disease. The S gene is a host gene that promotes the successful infection of pathogens, and mutations thereof may lead to specific resistance of a plant host to the pathogens, which may be passed on to future generations. Therefore, identification and destruction of Huanglongbing susceptibility genes are an attractive option for citrus disease resistance breeding.

Protein degradation in organisms occurs mainly through two mechanisms: a ubiquitin-proteasome system (UPS) and an autophagy-lysosome pathway. The ubiquitin-proteasome system is one of the common degradation mechanisms in cells, and E3 ligase is a key component of the ubiquitin-proteasome system. The system determines the specificity of a substrate and mediates the ubiquitination and degradation of the substrate. A plant U-box protein (PUB), as a member of the plant-specific E3 ligase family, plays a key role in determining the specificity of protein degradation of the substrate. It has been found through researches that proteins of the PUB family play a key role in interactions between rice, Arabidopsis, tobacco as well as tomato and pathogens. At the same time, the PUB E3 ligase also plays important roles in various signaling pathways that regulate plant immune responses and defense against pathogen infections, and therefore pathogen effectors often target PUBs and substrates thereof to suppress plant immunity.

### SUMMARY OF THE INVENTION

The technical problem to be solved by the present disclosure is how to regulate and control Huanglongbing resistance of a plant so as to cultivate a Huanglongbing-resistant plant. The technical problem to be solved is not limited to the described technical subject matter, and other technical subject matter not mentioned herein can be clearly understood by those skilled in the art by the following description.

In order to solve the technical problems described above, the present disclosure first provides novel uses of a CsPUB21 protein or a biomaterial related thereto.

The present disclosure provides application of a CsPUB21 protein or a biomaterial related thereto in any one of the following A1)-A3):
A1) regulation and controlling of plant resistance to Huanglongbing;
A2) regulation and controlling of insect resistance of a plant; or
A3) plant breeding, wherein
the CsPUB21 protein is any one of the following a1)-a4):
a1) a protein with an amino acid sequence of SEQ ID No. 1;
a2) a fused protein obtained by ligating a protein-tag at an N-terminus and/or a C-terminus of the amino acid sequence described in a1);
a3) a protein with the same function obtained by subjecting the amino acid sequence in a1) to substitution and/or deletion and/or addition of one or several amino acid residues; or
a4) a protein having 75% or more identity to the amino acid sequence in a1) and having the same function; and
the biomaterial is a nucleic acid molecule coding the CsPUB21 protein above or an expression cassette, a recombinant vector, a recombinant microorganism or a recombinant host cell containing the nucleic acid molecule.

In the protein described in a2) above, the protein-tag refers to a polypeptide or protein subjected to fused expression with a target protein using a DNA in vitro recombination technology, so as to facilitate expression, detection, tracing or purification of the target protein. Specifically, the protein-tag may be a Flag tag, a His tag, an MBP tag, an HA tag, a myc tag, a GST tag and/or a SUMO tag, etc.

In the protein described in a3) above, the substitution and/or deletion and/or addition of the one or several amino acid residues may specifically be the substitution and/or deletion and/or addition of no more than 10 amino acid residues, or the substitution and/or deletion and/or addition of no more than 9 amino acid residues, or the substitution and/or deletion and/or addition of no more than 8 amino acid residues, or the substitution and/or deletion and/or addition of no more than 7 amino acid residues, or the substitution and/or deletion and/or addition of no more than 6 amino acid residues, or the substitution and/or deletion and/or addition of no more than 5 amino acid residues, or the substitution and/or deletion and/or addition of no more than 4 amino acid residues, or the substitution and/or deletion and/or addition of no more than 3 amino acid residues, or the substitution and/or deletion and/or addition of no more than 2 amino acid residues and/or addition, or the substitution and/or deletion and/or addition of no more than 1 amino acid residue.

In the protein described in a4) above, the identity refers to the identity of amino acid sequences. The identity of the amino acid sequences may be determined using homology search sites on the Internet, such as a BLAST page on a NCBI home page. For example, the identity of a pair of amino acid sequences may be calculated in advanced BLAST 2.1 by using blastp as a program, setting an Expect value to 10, setting all Filters to OFF, using BLOSUM62 as a Matrix, setting Gap existence cost, Per residue gap cost, and Lambda ratio to 11, 1 and 0.85 (default value) respectively, and carrying out retrieval, and then a value of the identity (%) may be obtained. The identity includes an amino acid sequence having 75% or higher, or 80% or higher, or 85% or higher, or 90% or higher, or 91% or higher, or 92% or higher, or 93% or higher, or 94% or higher, or 95% or higher, or 96% or higher, or 97% or higher, or 98% or higher, or 99% or higher identity to the amino acid sequence shown in SEQ ID No.1 in the present disclosure.

Any one of the proteins described in a1)-a4) above may be synthesized artificially, or may be obtained by synthesizing coding genes thereof first and then performing biological expression.

Further, the nucleic acid molecule is any one of the following m1) and m2):
m1) a DNA molecule with a nucleotide sequence of SEQ ID No. 2; or
m2) a DNA molecule having 75% or more identity to the nucleotide sequence in m1) and coding the CsPUB21 protein described above.

The nucleic acid molecule described in m1) above may be DNA, such as cDNA, genomic DNA or recombinant DNA; and the nucleic acid molecule may also be RNA, such as mRNA or hnRNA.

In the nucleic acid molecule described in m2) above, the identity refers to sequence similarity to a natural nucleic acid sequence. The identity may be evaluated with naked eyes or with computer software. In the case of using the computer software, the identity between two or more sequences may be expressed as a percentage (%) which may be used to evaluate the identity between related sequences. The identity includes nucleotide sequences having 75% or higher, or 80% or higher, or 85% or higher, or 90% or higher, or 95% or higher identity to the nucleotide sequence coding a protein composed of the amino acid sequence shown in SEQ ID No. 1 of the present disclosure.

One of ordinary skill in the art can easily mutate the nucleotide sequence coding the above CsPUB21 protein of the present disclosure using known methods, such as directed evolution and point mutation. Those nucleotides which have been artificially modified to have 75% or higher identity to the CsPUB21 nucleotide sequence obtained in the present disclosure by separation, provided that they encode the CsPUB21 protein described above and have the same function, are nucleotide sequences derived from and equivalent to the sequences of the present disclosure.

In the above application, regulation and controlling of the Huanglongbing resistance of the plant is to reduce the Huanglongbing resistance of the plant.

Regulation and controlling the insect resistance of the plant is to reduce the insect resistance of the plant.

The purpose of plant breeding is to cultivate plants with reduced resistance to Huanglongbing.

The CsPUB21 protein of the present disclosure has the E3 ubiquitin ligase activity, and can inhibit synthesis of terpenes, such as β-caryophyllene, 7-epi-sesquiterpene, (E)-β-farnesene, and sesquiterpene through direct ubiquitination and degradation of a citrus Huanglongbing resistance-related protein CsMYC2, and attenuate a defense response of citrus based on a terpene compound, so that the attraction to Diaphorina citri Kuwayama is enhanced, and the replication and spread of Huanglongbing are promoted.

In order to solve the technical problems described above, the present disclosure further provides new uses of a substance that inhibits activity of a CsPUB21 protein or a substance that reduces a content of the CsPUB21 protein.

The present disclosure provides application of a substance that inhibits activity of a CsPUB21 protein or a substance that reduces a content of the CsPUB21 protein in any one of the following B1)-B5):
B1) improving Huanglongbing resistance of a plant;
B2) improving insect resistance of a plant;
B3) cultivating a Huanglongbing-resistant plant;
B4) cultivating an insect-resistant plant; or
B5) preventing and controlling Huanglongbing for a plant.

Further, the substance that inhibits the activity of the CsPUB21 protein may be a protein, a polypeptide or a small molecule compound that inhibits a function of the CsPUB21 protein.

The substance that reduces the content of the CsPUB21 protein may be a substance that inhibits synthesis of the CsPUB21 protein or promotes degradation of the CsPUB21 protein or knocks out or knocks down a CsPUB21 gene.

The substance knocking out the CsPUB21 gene may be a substance that achieves in any way that a host cell does not produce functional protein products of the CsPUB21 gene, such as by removing all or part of coding gene sequences, by introducing a frameshift mutation that results in the non-production of the functional protein, by removing or altering a regulatory component (e.g., promoter editing) so that the coding gene sequence is not transcribed, by preventing translation through binding to mRNA, and by mutating a key functional site. Typically, knockout is performed at a genomic DNA level, such that the progeny of cells are also permanently knocked out.

The substance knocking down the CsPUB21 gene may be a substance that inactivates the expression of the CsPUB21 gene or silences the gene from a transcriptional level or from a translational level in any way, such as RNA interference, Morpholino interference, antisense nucleic acids, nuclease enzymes, dominant negative inhibitory mutations, inhibition of the expression of the CsPUB21 gene by using shRNA or siRNA expressed by a virus (e.g., lentivirus, adenoassociated virus), etc.

Still further, the substance knocking out the CsPUB21 gene is any substance capable of inactivating the CsPUB21 gene by mutating (the mutation may be in a form of a deletion mutation and/or an insertion mutation and/or a base substitution) the CsPUB21 gene.

The substance knocking down the CsPUB21 gene is a fragment that silences the CsPUB21 gene.

In one specific implementation of the present disclosure, the substance knocking out the CsPUB21 gene is a prime editing vector for editing a codon TGC coding cysteine at position 39 of the CsPUB21 protein above to a codon GCC coding alanine.

In one specific implementation of the present disclosure, the fragment that silences the CsPUB21 gene is a nucleic acid molecule shown in SEQ ID No. 3.

In another specific implementation of the present disclosure, the fragment that silences the CsPUB21 gene is a nucleic acid molecule shown in SEQ ID No. 4.

In order to solve the technical problems described above, the present disclosure further provides a CsPUB21 mutant protein (denoted as CsPUB21DN).

The CsPUB21 mutant protein provided by the present disclosure is a protein obtained by mutating the cysteine at position 39 of the amino acid sequence of the CsPUB21 protein above to alanine.

In one specific implementation of the present disclosure, the amino acid sequence of CsPUB21DN is as shown in SEQ ID No. 5.

The biomaterial related to the CsPUB21 mutant protein above also falls within the scope of protection of the present disclosure.

The biomaterial is a nucleic acid molecule coding the CsPUB21DN protein above or an expression cassette, a recombinant vector, a recombinant microorganism or a recombinant host cell containing the nucleic acid molecule.

Further, the nucleic acid molecule is any one of the following n1) and n2):
n1) a DNA molecule with a nucleotide sequence of SEQ ID No. 6; or
n2) a DNA molecule having 75% or more identity to the nucleotide sequence in n1) and coding the CsPUB21DN protein described above.

The nucleic acid molecule described in n1) above may be DNA, such as cDNA, genomic DNA or recombinant DNA; and the nucleic acid molecule may also be RNA, such as mRNA or hnRNA.

In the nucleic acid molecule described in n2) above, the identity refers to sequence similarity to a natural nucleic acid sequence. The identity may be evaluated with naked eyes or with computer software. In the case of using the computer software, the identity between two or more sequences may be expressed as a percentage (%) which may be used to evaluate the identity between related sequences. The identity includes nucleotide sequences having 75% or higher, or 80% or higher, or 85% or higher, or 90% or higher, or 95% or higher identity to the nucleotide sequence coding a protein composed of the amino acid sequence shown in SEQ ID No. 5 of the present disclosure.

Those ordinarily skilled in the art can easily mutate the nucleotide sequence coding the above CsPUB21DN protein of the present disclosure using known methods, such as directed evolution and point mutation. Those nucleotides which have been artificially modified to have 75% or higher identity to the nucleotide sequence of the CsPUB21DN protein obtained in the present disclosure by separation, provided that they encode the CsPUB21DN protein described above and have the same function, are nucleotide sequences derived from the present disclosure and equivalent to the sequences of the present disclosure.

The CsPUB21DN protein of the present disclosure is a naturally present PUB21 dominant negative mutant CsPUB21DN (a highly conserved site shown at position 39 in the amino acid sequence of the CsPUB21 protein is mutated from the cysteine Cys to the alanine Ala) found in the rutaceae plants, which can block the E3 ligase activity of CsPUB21, promote synthesis of terpene compounds (e.g., β-caryophyllene, 7-epi-sesquiterpene, (E)-β-farnesene, and sesquiterpene), and enhance the defense response of citrus based on the terpene compound, so that avoidance of Diaphorina citri Kuwayama is enhanced, and the replication and spread of Huanglongbing are inhibited.

In order to solve the technical problems described above, the present disclosure further provides novel uses of the CsPUB21DN protein described above or a biomaterial related thereto.

The present disclosure provides application of the CsPUB21DN protein above or a biomaterial related thereto in any one of the following C1)-C5):
C1) improving Huanglongbing resistance of a plant;
C2) improving insect resistance of a plant;
C3) cultivating a Huanglongbing-resistant plant;
C4) cultivating an insect-resistant plant; or
C5) preventing and controlling Huanglongbing for a plant.

Any one of the expression cassettes above may be DNA capable of expressing the CsPUB21 or CsPUB21DN protein above in a host cell. The DNA may include not only a promoter that initiates transcription of a CsPUB21 or CsPUB21DN gene, but also a terminator that terminates transcription of the CsPUB21 or CsPUB21DN gene. The expression cassette may further include an enhancer sequence.

Any one of the vectors above refers to a vector capable of transporting the CsPUB21 or CsPUB21DN gene into a host cell for amplification and expression. The vector may be a cloning vector or an expression vector, including but not limited to: plasmid, phage (e.g., λ-phage or M13 filamentous phage), kozmidy (i.e., cosmid), Ti plasmid, and a viral vector (e.g., retrovirus (including lentivirus), adenovirus, and adeno-associated virus). The recombinant vector refers to a recombinant DNA molecule constructed by ligating the CsPUB21 or CsPUB21DN gene to a vector in vitro, specifically may be a vector containing a cassette of the CsPUB21 or CsPUB21DN gene and constructed with existing plant expression vectors. The plant expression vectors include binary Agrobacterium vectors and vectors that may be used for plant gene gun transformation, etc., such as pAHC25, pBin438, pCAMBIA1302, pCAMBIA2301, pCAMBIA1301, pCAMBIA1300, pBI121, pCAMBIA1391-Xa or pCAMBIA1391-Xb. The plant expression vectors may also include a 3′-end untranslated region of an exogenous gene, i.e. including a polyadenylate signal and any other DNA fragment involved in mRNA treatment or gene expression. The polyadenylate signal may direct the incorporation of polyadenylate into the 3′ end of an mRNA precursor, e.g., Agrobacterium crown gall tumor-induced (Ti) plasmid genes (e.g., a nopaline synthase gene, Nos), and an untranslated region of transcription at the 3′ end of plant genes (e.g., soybean storage protein genes) all have similar functions. When plant expression vectors are constructed using the genes of the present disclosure, enhancers, including translational enhancers or transcriptional enhancers, may also be used, and these enhancer regions may be ATG start codons or neighboring region start codons, etc., and have the same reading frame as a coding sequence so as to ensure the correct translation of the entire sequence. The sources of the translation control signals and start codons are wide-ranging and may be natural or synthetic. A translation start region may be derived from a transcription start region or a structural gene. In order to facilitate the identification and screening of transgenic plant cells or plants, the plant expression vectors used may be processed, such as adding genes (GUS genes, luciferase genes, etc.) that may be expressed in plants and are used for coding enzymes that may produce color changes or luminous compounds, antibiotic marker genes (such as nptII genes conferring resistance to kanamycin and related antibiotics, bar genes conferring resistance to herbicide phosphonisin, hph genes conferring resistance to antibiotic hygromycin, dhfr genes conferring resistance to amethopterin, and EPSPS genes conferring resistance to glyphosate), or chemical reagent resistant marker genes (such as herbicide resistance genes), and mannose-6-phosphate isomerase genes providing the mannose metabolization capability. Considering the safety of transgenic plants, transformed plants may be screened directly under stress without any selective marker genes.

Any one of the microorganisms described above may be bacteria, fungi, actinomyces, protozoa, algae or viruses. The bacteria may be derived from, but not limited to, Escherichia sp., Erwinia sp., Agrobacterium sp., Flavobacterium sp., Alcaligenes sp., Pseudomonas sp., Bacillus sp., etc., for example, the bacteria may be Escherichia coli, Bacillus subtilis, or Bacillus pumilus. The fungi may be yeast, and the yeast may be derived from, but not limited to, Saccharomyces sp. (Such as Saccharomyces cerevisiae), Kluyveromyces sp. (such as Kluyveromyces lactis), Pichia sp. (such as Pichia pastoris), Schizosaccharomyces sp. (Such as Schizosaccharomyces pombe), and Hansenula sp. (such as Hansenula polymorpha). The fungi may also be derived from, but not limited to, Fusarium sp., Rhizoctonia sp., Verticillium sp., Penicillium sp., Aspergillus sp., and Cephalosporium sp. The actinomyces may be derived from, but not limited to, Streptomyces sp., Nocardia sp., Micromonospora sp., Streptosporangium sp., Actinoplanes sp., and Thermoactinomyces sp. The algae may be derived from, but not limited to, Fucus sp., Achnanthes sp., Amphiprora sp., Amphora sp., Ankistrodesmus sp.), Asteromonas sp., and Boekelovia sp. The viruses may be, but not limited to, rotavirus, herpes virus, influenza virus, and adenovirus.

Any one of the above host cells may be a plant cell or an animal cell. The host cell may be understood to refer not only to a specific recipient cell, but also to progeny of such cell that, and due to natural, accidental or intentional mutations and/or alterations, the progeny may not have to be exactly identical to an original parent cell, but still be included in the range of the host cell. Suitable host cells are known in the art, where the plant cells may be, but not limited to, Arabidopsis thaliana, Nicotiana tabacum, Zea mays, Oryza sativa, and Triticum aestivum; and the animal cells may be, but not limited to, mammalian cells (such as Chinese hamster ovary cells (CHO cells), African green monkey kidney cells (Vero cells), baby hamster kidney cells (BHK cells), mouse mammary tumor cells (C127 cells), human embryonic kidney cells (HEK293 cells), human HeLa cells, fibroblasts, myeloid cell series, T cells, or NK cells), avian cells (e.g., chicken or duck cells), amphibian cells (such as Xenopus laevis cells or Andrias davidianus cells), fish cells (such as grass carp, carp, rainbow trout or catfish cells), and insect cells (such as Sf21 cells or Sf-9 cells).

Any one of the recombinant microorganisms (or recombinant host cells) described above refers to a recombinant microorganism with functions changed (or a recombinant host cell with functions changed) obtained by performing operation and modification on genes of a target microorganism (or target host cell). For example, the recombinant microorganism is a recombinant microorganism (or a recombinant host cell) obtained after introducing the CsPUB21 or CsPUB21DN gene or an expression cassette or recombinant vector containing the CsPUB21 or CsPUB21DN gene into the target microorganism (or the target host cell). The recombinant microorganism (or the recombinant host cell) may be understood to refer not only to a specific recombinant microorganism (or recombinant host cell), but also to a descendant of such cell that, and due to natural, accidental or intentional mutations and/or alterations, the progeny may not have to be exactly identical to the original parent cell, but still be included in the scope of the recombinant microorganism (or the recombinant host cell).

In order to solve the technical problems described above, the present disclosure further provides a method for cultivating a Huanglongbing-resistant plant.

The method for cultivating the Huanglongbing-resistant plant provided by the present disclosure is any one of the following X1), or X2) or X3):
X1) reducing an expression and/or activity of the CsPUB21 protein described above in a recipient plant to obtain the Huanglongbing-resistant plant;
X2) editing a codon coding cysteine at position 39 of the CsPUB21 protein described above in the recipient plant to a codon coding other amino acids to obtain the Huanglongbing-resistant plant; or
X3) improving the expression and/or activity of a CsPUB21 mutant protein described above in the recipient plant to obtain the Huanglongbing-resistant plant.

In order to solve the technical problems described above, the present disclosure further provides a method for cultivating an insect-resistant plant.

The method for cultivating the insect-resistant plant provided by the present disclosure is any one of the following Y1), or Y2) or Y3):
Y1) reducing an expression and/or activity of the CsPUB21 protein described above in a recipient plant to obtain the insect-resistant plant;
Y2) editing a codon coding cysteine at position 39 of the CsPUB21 protein described above in the recipient plant to a codon coding other amino acids to obtain the insect-resistant plant; or
Y3) improving the expression and/or activity of the CsPUB21 mutant protein described above in the recipient plant to obtain the insect-resistant plant.

In the methods described as (X1) or (X2) or (Y1) or (Y2), the recipient plant contains the CsPUB21 protein described above.

In any one of the above-described methods, the method for reducing the expression and/or activity of the CsPUB21 protein in the recipient plant is to introduce into the recipient plant a substance that inhibits the activity of the CsPUB21 protein or a substance that reduces a content of the CsPUB21 protein described above.

Further, the substance that reduces the content of the CsPUB21 protein is a substance that inhibits the expression of a CsPUB21 gene.

Furthermore, the substance that inhibits the expression of the CsPUB21 gene is a fragment that silences the CsPUB21 gene.

Still further, the fragment that silences the CsPUB21 gene is a nucleic acid molecule shown in SEQ ID No. 3 or SEQ ID No. 4.

In one specific implementation of the present disclosure, the substance that reduces the content of the CsPUB21 protein is a gene silencing vector CsPUB21 RNAi-1 or a gene silencing vector CsPUB21 RNAi-2 in the embodiment below.

In any one of the methods described above, the other amino acids are alanine.

In any one of the methods described above, editing the codon coding cysteine at position 39 of the CsPUB21 protein above in the recipient plant to the codon coding other amino acids is to edit the codon coding cysteine at position 39 of the CsPUB21 protein above in the recipient plant to a codon coding alanine.

Further, editing the codon coding cysteine at position 39 of the CsPUB21 protein above in the recipient plant to the codon coding alanine is to edit a codon TGC coding cysteine at position 39 of the CsPUB21 protein above in the recipient plant to a codon GCC coding alanine.

Furthermore, a method for editing the codon TGC coding cysteine at position 39 of the CsPUB21 protein above in the recipient plant to the codon GCC coding alanine is to introduce a prime editing vector of a codon targeting the amino acid at position 39 of the CsPUB21 protein above into the recipient plant.

Still further, the prime editing vector of the codon targeting the amino acid at position 39 of the CsPUB21 protein described above expresses pegRNA (the pegRNA includes a target sequence, an esgRNA backbone, an RT sequence, and a PBS sequence in sequence) of the codon targeting the amino acid at position 39 of the CsPUB21 protein above, and a fused protein formed by fusion of nCas9 (H840A) and M-MLV.

In one specific implementation of the present disclosure, the prime editing vector of the codon targeting the amino acid at position 39 of the CsPUB21 protein above is a PE2(Cs)-peg-CsPUB21 prime editing vector in the embodiment below. In the PE2(Cs)-peg-CsPUB21 prime editing vector, the pegRNA is driven by a CsU6 promoter (as shown in SEQ ID No. 35) for expression; and the fused protein is driven by a CmYLCV promoter (as shown in SEQ ID No. 36) for expression, and includes a target sequence, an esgRNA backbone, an RT sequence and a PBS sequence in sequence, the target sequence is as shown in SEQ ID No. 81, and the RT sequence and the PBS sequence are shown in SEQ ID No. 82.

In any one of the methods described above, the method for improving the expression and/or activity of the CsPUB21DN protein in the recipient plant is overexpression of the CsPUB21DN protein in the recipient plant. Further, the method of overexpression is to introduce a gene coding the CsPUB21DN protein above into the recipient plant. Furthermore, the gene coding the CsPUB21DN protein is as shown in SEQ ID No. 6.

In one specific implementation of the present disclosure, the gene coding the CsPUB21DN protein passes through a recombinant vector CsPUB21DN-pLGN135 in the embodiment below.

In any one of the methods described above, the introduction manner includes, but is not limited to: transfecting plant cells or tissues by using conventional biological methods such as Ti plasmids, Ri plasmids, plant viral vectors, direct DNA transformation, microinjection, electroconduction, Agrobacterium mediation, etc., and cultivating the transfected plant cells or tissues into plants.

In any one of the methods described above, the amount (concentration or titer) of Huanglongbing pathogens CLas in leaves of the Huanglongbing-resistant plant is lower than that of the recipient plant. Leaves of the insect-resistant plant attract less Diaphorina citri Kuwayama than the recipient plant.

In any one of the applications or methods described above, the Huanglongbing resistance is resistance to Huanglongbing pathogens. The Huanglongbing pathogens may specifically be a Huanglongbing pathogen Clas.

In any one of the applications or methods described above, the insect resistance is resistance to Diaphorina citri Kuwayama.

In any one of the applications or methods described above, the plant is any one of the following P1)-P5):
P1) monocotyledonous plants or dicotyledonous plants;
P2) rutales plants;
P3) rutaceae plants;
P4) citrus plants; or
P5) citrus (such as Huanglongbing-susceptible Citrus sinensis L. and Valencia orange).

The Huanglongbing resistant plant or the insect resistant plant produced by any one of the methods described above also falls within the scope of protection of the present disclosure.

The present disclosure identifies that the CsPUB21 protein has the E3 ubiquitin ligase activity, and attenuates the defense response of a citrus plant based on the terpene compound through direct ubiquitination and degradation of a citrus Huanglongbing resistance-related protein CsMYC2, and the resistance of citrus to the Huanglongbing pathogen-CLas and a spread vector thereof-Asian Diaphorina citri Kuwayama, can be significantly improved by silencing of the CsPUB21 gene. Meanwhile, the present disclosure identifies a naturally present PUB21 dominant negative mutant CsPUB21DN (highly conserved Cys-39 mutated to Ala), which can block the E3 ubiquitin ligase activity of the CsPUB21 protein and has effects of resisting the Huanglongbing pathogen CLas and Diaphorina citri Kuwayama. Further, according to the present disclosure, the mutation of the amino acid Cys to Ala is artificially realized by replacing the codon TGC coding an amino acid at position 39 of a key enzyme activity site of the CsPUB21 protein in a disease-susceptible plant with GCC by a prime editing technique, and the active form of CsPUB21DN is formed, and the concentration of the Huanglongbing pathogen CLas in the disease-susceptible plant is effectively reduced.

### BRIEF DESCRIPTION OF THE DRAWINGS

FIG. 1A-D is a relative expression level of CsPUB21 and a relative concentration of a pathogen CLas in Huanglongbing susceptible Citrus sinensis L. leaves that silences a CsPUB21 gene. FIG. 1A shows phenotypes of healthy Citrus sinensis L. leaves and Huanglongbing susceptible Citrus sinensis L. leaves. FIG. 1B shows molecular identification results of healthy Citrus sinensis L. leaves and Huanglongbing susceptible Citrus sinensis L. leaves. FIG. 1C shows a relative expression level of CsPUB21 in Huanglongbing susceptible Citrus sinensis L. leaves that silences a CsPUB21 gene. Values are means±SEM (n=9) (**P<0.01, Student's t-test). FIG. 1D shows a relative concentration of a pathogen CLas in Huanglongbing susceptible Citrus sinensis L. leaves that silences a CsPUB21 gene (CsPUB21 RNAi) or overexpresses CsPUB21DN. Data represents means±SEM (n=9), and lowercase letters indicates significant differences (P<0.05) between different columns according to one-way analysis of variance and Duncan multi-range test.

FIG. 2A-C is discovery and analysis of a dominant negative mutant CsPUB21DN of PUB21. FIG. 2A shows two PUB21 genotypes (a wild PUB21 genotype: CACTTCCGGTGCCCGATA (SEQ ID No. 68), and a dominant negative PUB21^{C39A} mutant genotype (PUB21DN): CACTTCCGGGCCCCGATA (SEQ ID No. 69)) in rutaceae plants. A PCR amplicon sequence shows that there are two mutated bases (red) near a length of 120 bp containing a site Cys39 and a dominant negative mutant CsPUB21DN (Cys-39 mutated to Ala, PUB21^{C39A}) of wild-type PUB21 is produced. FIG. 2B shows amino acid sequences of U-box structural domains of PUB proteins from different species (CsPUB2: APSHFFCPILQEVMEDPYIAADGFTYEHRAIKAWLEKH (SEQ ID No. 70); CsPUB9: CPEEFKCPLSKELMRDPVILASGQTFDRPYIQRWLKAGN (SEQ ID No. 71); CsPUB10: IPADFLCPISLELMRDPVIVATGQTYERSYIQRWIDCGN (SEQ ID No. 72); CsPUB16: VPADFRCPISLELMRNPVVVATGQTYDRQSISLWIESGH (SEQ ID No. 73); CsPUB21, MaPUB21, MkPUB21, ZbPUB21 TPNHFRCPISLDLMKDPVTLSTGITYDRENIEKWIHEDGN (SEQ ID No. 74); MpPUB21, RgPUB21: TPNHFRCPISLDLMKDPVTLSTGITYDRENIEKWIHENGN (SEQ ID No. 75); AtPUB21: IPPEFQCPISIDLMKDPVIISTGITYDRVSIETWINSGN (SEQ ID No. 76); GhPUB21: VPRDFRCPISLDLMKDPVTLSTGITYDRENIEKWIEAGN (SEQ ID No. 77); SlPUB21: VPSHFKCPISLDLMSPDPVTLSTGITYDRVSIETWIENGN (SEQ ID No. 78); MpPUB21DN, MkPUB21DN, RgPUB21DN, ZbPUB21DN: TPNHFRAPISLDLMKDPVTLSTGITYDRENIEKWIHEDGN (SEQ ID No. 79)). Numbers refer to a position of a first amino acid in the sequence, and asterisks indicate conserved cysteine residues. Cs: Citrus sinensis; Mp: Murraya paniculata; Mk: Murraya koenigii; Ma: Microcitrus australiasica; Zb: Zanthoxylum bungeanum; Rg: Rutagraveolens; At: Arabidopsis thaliana; Gh: Gossypium hirsutum; and Sl: Solanum lycopersicum. FIG. 2C shows in vitro auto-ubiquitination of citrus CsPUB21, where E3 ligase activity of CsPUB21DN is blocked by a mutation thereof at a conserved site in a U-box structural domain, and an anti-c-myc tag antibody is used for an immunoblotting reaction.

FIG. 3A-B is analysis of relative expression levels of a PUB21 gene and a PUB21DN gene in different rutaceae species. FIG. 3A shows relative expression levels of a PUB21 gene in different rutaceae species. Data represents means±SEM (n=4), and lowercase letters indicate significant differences (P<0.05) between different columns according to one-way analysis of variance and Duncan multi-range test. FIG. 3B shows relative expression percentages of a PUB21 gene and a PUB21DN gene in different rutaceae species.

FIG. 4A-C is a partial illustration of PE2(Cs)-peg-CsPUB21 prime editing, analysis of efficiency of CsPUB21 prime editing and a relative titer of a pathogen CLas in Huanglongbing susceptible protoplast that edits a CsPUB21 gene. FIG. 4A shows a partial illustration of a CsPUB21 gene locus and pegRNA design in PE2(V2) and PE2(Cs) constructs (Wild type: AACCACTTCCGGTGCCCGATATCTCTCGACT (SEQ ID No. 80); pegRNA sgRNA site: CCGGTGCCCGATATCTCTCGACT (SEQ ID No. 81); RT+PBS: AACCACTTCCGGGCCCCGATATCT (SEQ ID No. 82)). PAM and required inserts are marked in red and blue, respectively. FIG. 4B shows plasmid editing efficiency of an original editing vector PE2(V2) and a promoter-optimized vector PE2(Cs) at a peg-CsPUB21 site in citrus protoplast. FIG. 4C shows relative titers of CLas in HLB-positive citrus protoplast transfected with a PE2(Cs) blank vector and PE2(Cs)-peg-CsPUB21. Data indicates means±SEM (n=3), and lowercase letters indicate significant differences (P<0.05) between different columns according to one-way analysis of variance and Duncan multi-range test.

FIG. 5A-B is phenotypes of transgenic citrus overexpressing CsPUB21 and CsPUB21DN and a relative expression level of CsPUB21 in leaves. FIG. 5A shows phenotypes of vector control citrus, and transgenic citrus overexpressing CsPUB21 and CsPUB21DN. FIG. 5B shows relative expression levels of CsPUB21 in vector control citrus, and transgenic citrus overexpressing CsPUB21 and CsPUB21DN. Values are means±SEM (n=3) (**P<0.01, Student's t-test).

FIG. 6A-D shows that CsPUB21 promotes replication and spread of citrus Huanglongbing by inhibiting synthesis of β-caryophyllene. FIG. 6A shows a selection preference of Diaphorina citri Kuwayama for transgenic citrus leaves overexpressing CsPUB21 and CsPUB21DN, with citrus leaves overexpressing a blank vector as a control. Values are means±SEM (n=8) (** P<0.05, Wilcoxon paired test). FIG. 6B shows a relative gene expression level of a terpene synthase CsTPS21 gene cluster in blank vector control citrus leaves and in transgenic citrus leaves overexpressing CsPUB21. Citrus leaves overexpressing a blank vector are used as a control. Values are means±SEM (n=4) (ns indicating no significant difference; *, P<0.05; **, P<0.01; Student's t-test). FIG. 6C shows a GC-MS chromatogram of a product obtained by catalyzing FPP with purified GST and GST-CsTPS21-7 proteins, where 1, 7-epi-sesquiterpene; 2, (E)-β-farnesene; 3, β-caryophyllene; and 4, sesquiterpene. FIG. 6D shows a selection preference of Diaphorina citri Kuwayama for (E)-β-farnesene or β-caryophyllene, with a solvent (n-hexane) as a control. Values are means±SEM (n=8) (**P<0.01, Wilcoxon paired test).

FIG. 7A-C shows that a citrus Huanglongbing resistance-related protein MYC2 is a direct acting substrate of PUB21. FIG. 7A shows CsPUB21 full-length and deletion mutants. FIG. 7B shows presence of an interaction between CsPUB21 and CsMYC2 in a yeast two-hybrid system. A yeast strain AH109 co-transformed with plasmid shown is found on an SD-Leu-Trp-His medium containing 5 mM 3-AT. Blank vectors pGAD424 (AD) and pGBDT7 (BD) are used as negative controls. FIG. 7C is BiFC analysis to clarify an interaction between CsMYC2 and CsPUB21 and deletion mutants thereof. The scale represents 50 μm.

FIG. 8A-D is direct ubiquitination and degradation of MYC2 with a Huanglongbing pathogen resistance effect by CsPUB21. FIG. 8A shows that CsPUB21 is able to directly polyubiquitinate CsMYC2 in vitro. The level of polyubiquitination of an MBP-MYC2 protein is tested in the presence of AtUBA1, AtUBC8, CsPUB21 and ubiquitin, and a CsPUB21DN mutant protein is used as a negative control. FIG. 8B shows an effect of CsPUB21 on stability of a CsMYC2 protein in plants. 35S:CsMYC2-myc is co-injected with 35S:YFP-CsPUB21, 35S:YFP-CsPUB21DN, or the negative control 35S:YFP into tobacco plant leaves for expression of 48 h, and a cumulative level of the CsMYC2 protein co-expressed with a YFP protein is set to 1. FIG. 8C shows that a relative concentration of CLas in Huanglongbing susceptible Citrus sinensis L. leaves overexpressing YFP and CsMYC2 is determined by qPCR. Data represents means±SEM (n=9) (**P<0.01, Student's t-test). FIG. 8D shows changes in terpene volatiles of citrus after silencing a citrus CsMYC2 gene. Values are means±SEM (n=3) (ns indicates no significant difference; **P<0.01, Student's t-test).

FIG. 9A-C shows phenotypes and pathogen titers of transgenic citrus overexpressing CsPUB21 and CsPUB21DN to infestation with a Huanglongbing pathogen CLas. FIG. 9A shows disease phenotypes of vector control citrus and transgenic citrus overexpressing CsPUB21 and CsPUB21DN 3 months after grafting branches infected with a Huanglongbing pathogen CLas onto transgenic citrus plants. The scale length is 3 cm. FIG. 9B shows the quantification of CLas titers in transgenic citrus leaves using qPCR 2 and 3 months after grafting branches infected with a Huanglongbing pathogen CLas onto vector control citrus and transgenic citrus overexpressing CsPUB21 and CsPUB21DN. Values are means±SEM (n=3) (ns indicates no significant difference; *P<0.05; **P<0.01, Student's t-test). FIG. 9C shows that Diaphorina citri Kuwayama carrying a Huanglongbing pathogen CLas delivers the pathogen to vector control and transgenic citrus overexpressing CsPUB21DN by feeding, and the CLas titer in plants is quantified after 7 days using qPCR. Values are means±SEM (n=6) (ns indicates no significant difference; **P<0.01, Student's t-test).

### DETAILED DESCRIPTION

The present disclosure is described in further detail below in conjunction with specific embodiments, and the embodiments given are intended only to clarify the present disclosure and not to limit the scope of the present disclosure. The following embodiments are provided as a guide for further improvement by a person of ordinary skill in the art, and do not in any way constitute a limitation on the present disclosure.

The experimental methods in the following embodiments, if not otherwise specified, are conventional methods and are carried out in accordance with the techniques or conditions described in the literature in the field or in accordance with the product specification. The materials, reagents, and the like used in the following embodiments are commercially available, if not otherwise specified.

A pENTR-3C vector in the following embodiments is a product of Invitrogen, and an antibiotic used is a kanamycin antibiotic (Kan).

A PE2(V2) vector in the following embodiments is recorded in the literature “Zhen Liang, Yuqing Wu, Yingjie Guo, Sha We. Addition of the T5 exonuclease increases the prime editing efficiency in plant. Journal of Genetics and Genomic, 2023, 50:582-588”, and an antibiotic used is a kanamycin antibiotic (Kan).

A pH7-YFP-DC vector in the following embodiments is a product of Invitrogen, and an antibiotic used is spectinomycin (Spe).

A pBA-DC-myc vector in the following embodiments is a product of Invitrogen, and an antibiotic used is spectinomycin (Spe).

A pH7GWIWG2(II) vector in the following embodiments is a product of Invitrogen, and an antibiotic used is spectinomycin (Spe).

A pGEX-DC-3HA vector in the following embodiments is a product of Invitrogen, and an antibiotic used is spectinomycin (Spe).

A pET-28a(+) vector in the following embodiments is a product of Invitrogen, and an antibiotic used is spectinomycin (Spe).

A 35S:YFP control vector in the following embodiments is a vector obtained after cloning a YFP gene into the pBA-DC-myc vector.

Vectors pACYCDuet-CDS-Myc-AtUBC8-S, pCDFDuet-AtUBA1-S, pCDFDuet-MBP-DC-HA-AtUBA1-S, and pET-28a-FLAG-UBQ in the following embodiments are recorded in the literature “Yufang Han, Jianhang Sun, Jun Yang, Zhaoyun Tan, Jijing Luo, Dongping Lu. Reconstitution of the plant ubiquitination cascade in bacteria using a synthetic biology approach. The Plant Journal 91, 766-776 (2017)”.

Fluorescent complementary expression vectors pSAT1-cEYFP and pSAT1-nEYFP in the following embodiments are both recorded in the literature “Ran Li, Berhane T. Weldegergis, Jie Li, Choonkyun Jung, Jing Qu, Yanwei Sun, Hongmei Qian, Chuan Sia Tee Joop J. A. van Loon, Marcel Dicke, Nam-Hai Chua, Shu-Sheng Liu and Jian Ye. Virulence Factors of Geminivirus Interact with MYC2 to Subvert Plant Resistance and Promote Vector Performance. The plant cell, 2014.”.

A citrus transgenic vector pLGN135 in the following embodiments is recorded in the literature “Hongwei Shi, Zuhui Yang, Jie Huang, Haodi Wu, Shimin Fu, Weimin Li, Xiuping Zou, Changyong Zhou, Xuefeng Wang. An effector of ‘Candidatus Liberibacter asiaticus’ manipulates autophagy to promote bacterial infection. J Exp Bot, 2023, 74(15):4670-4684”.

As for rutaceae plants used in the following embodiments, Citrus sinensis L. is collected from Lianjiang, Guangdong Province; Citrus reticulate is collected from Nanning, Guangxi Province; Citrus maxima is collected from Danzhou, Hainan Province; Citrus sinensis L. Osbeck, Microcitrusaustraliasica, and Murrayakoenigii are all collected from a Citrus Resource Nursery of Citrus Research Institute of Southwest University, Chongqing; Clausenalansium (Lour.) Skeels, Glycosmiscitrifolia (Willd.) Lindl and Rutagraveolens L. are all collected from Guangzhou, Guangdong Province; and Zanthoxylum bungeanum Maxim and Ponciruspolyandra are both collected from Kunming, Yunnan Province; and Murrayapaniculata is obtained from Guangzhou, Guangdong Province. Citrus sinensis L. Osbeck, Citrus sinensis L., Citrus reticulate, Citrus maxima and Murrayapaniculata are grown in a laboratory greenhouse, where a temperature is maintained at 27° C. and a relative humidity is 50%.

A transgenic chassis material used in the following embodiments is Valencia orange, and a grafting rootstock is Citrus aurantium, both of which are provided by Citrus Research Institute of Southwest University, Chongqing.

Diaphorina citri Kuwayama used in the following embodiments is kept in an insect-rearing cage with Murrayapaniculata as a host plant for growth and reproduction, where a temperature in the greenhouse is 27° C., a relative humidity is 50%, and a light cycle is 12 h light/12 h darkness.

Principal reagents in the following embodiments and sources thereof are as follows:

Reagents used for molecular cloning: both EX Taq DNA polymerase and LA Taq DNA polymerase are products from Takara, restriction endonuclease and T4 DNA ligase are products from NEB, homologous recombination ligase Vazyme ClonExpressII One Step Cloning C112 is a product from Vazyme, antibiotics used are products from Inolco, 1 Kb DNA marker and 2000 bp DNA marker are products from Biomed, and SYBR qPCR Mix is a product from TOYOBO.

Reagents used in protein-related experiments: Cocktail protease inhibitor is a product from Roche, IPTG is a product from Inolco, 40% Acrylamide is a product from Sigma, primary and secondary antibodies are both products from Beijing Transgen Biotech, and pre-stained protein molecular weight marker is a product from Easybio.

Kits: plasmid mini prep kits, plasmid maxi kits, agarose gel DNA recovery kits, and DNA purification recovery kits are products from AxyGen, Plant RNA Mini Prep Kit is a product from Qiagen, ECL luminescent liquid is a product from GE healthcare, and a reverse transcription kit is a product from Beijing Transgen Biotech.

Primers used in the following embodiments are synthesized by Tsingke Biotech, and subjected to related sequencing.

An amino acid sequence of a CsPUB21 protein in the following embodiments is as shown in SEQ ID No. 1, and a CsPUB21 gene coding sequence (CDS) is as shown in SEQ ID No. 2.

An amino acid sequence of a CsPUB21DN protein in the following embodiments is as shown in SEQ ID No. 5, and a CsPUB21DN gene coding sequence (CDS) is as shown in SEQ ID No. 6.

An amino acid sequence of a CsTSP21-7 protein in the following embodiments is as shown in SEQ ID No. 7, and a CsTSP21-7 gene coding sequence (CDS) is as shown in SEQ ID No. 8.

An amino acid sequence of a CsMYC2 protein and a CsMYC2 gene coding sequence (CDS) in the following embodiments are recorded in an invention patent application file with a publication number CN115160421A.

### Embodiment 1: Resistance of Citrus to Huanglongbing Pathogen CLas can be Improved by Silencing CsPUB21

### I. Construction of a Citrus CsPUB21 Gene Silencing Vector

1. Gene fragment amplification primers are designed, the primer sequences being as follows:

CsPUB21-Fw (KpnI):
(SEQ ID No. 9)
CAAGGGTACCATGATTTTGTCATGGAAAAGACT;
CsPUB21-Rv (XhoI):
(SEQ ID No. 10)
CAAGCTCGAGAACGGCCTTTTCAGGTCCTT;
CsPUB21-606RNAi-Rv (XhoI):
(SEQ ID No. 11)
CAAGCTCGAGCAGCAACGTCAAAGTCGACA;
and
CsPUB21-800RNAi-Rv (XhoI):
(SEQ ID No. 12)
CAAGCTCGAGTTAAATAGCGGTTCAATGGC,

where nucleotide sequences shown underlined were an enzymatic recognition site GGTACC of KpnI and an enzymatic recognition site CTCGAG of XhoI.

2. With the cDNA of Citrus sinensis L.Osbeck as a template, PCR amplification carried out using primers CsPUB21-Fw (KpnI) and CsPUB21-Rv (XhoI) to obtain a CsPUB21 gene with a size of 1332 bp.

With the cDNA of Citrus sinensis L.Osbeck as a template, PCR amplification was carried out using primers CsPUB21-Fw (KpnI) and CsPUB21-606RNAi-Rv (XhoI) to obtain a CsPUB21 gene silencing fragment with a size of 606 bp, and a nucleotide sequence thereof was as shown in SEQ ID No.3.

With the cDNA of Citrus sinensis L.Osbeck as a template, PCR amplification was carried out using primers CsPUB21-Fw (KpnI) and CsPUB21-800RNAi-Rv (XhoI) to obtain a CsPUB21 gene silencing fragment with a size of 800 bp, and a nucleotide sequence thereof was as shown in SEQ ID No.4.

3. The pENTR-3C vector was double digested with restriction endonuclease KpnI and XhoI to obtain a backbone vector; and then the backbone vector was ligated, using T4 DNA ligase, to the CsPUB21 gene with the size of 1332 bp, the CsPUB21 gene silencing fragment with the size of 606 bp, and the CsPUB21 gene silencing fragment with the size of 800 bp in step 2, respectively, to obtain intermediate vectors pENTR-3C-CsPUB21, pENTR-3C-CsPUB21-RNAi-1 and pENTR-3C-CsPUB21-RNAi-2, respectively.

4. The intermediate vector pENTR-3C-CsPUB21 and an expression vector pH7-YFP-DC were ligated by an LR reaction using recombinase (Gateway LR Clonase II) to obtain recombinant plasmid 35S:YFP-CsPUB21.

The intermediate vector pENTR-3C-CsPUB21-RNAi-1 and an expression vector pH7GWIWG2(II) were ligated by the LR reaction using recombinase (Gateway LR Clonase II) to obtain a gene silencing vector CsPUB21 RNAi-1.

The intermediate vector pENTR-3C-CsPUB21-RNAi-2 and an expression vector pH7GWIWG2(II) were ligated by the LR reaction using recombinase (Gateway LR Clonase II) to obtain a gene silencing vector CsPUB21 RNAi-2.

### II. CsPUB21 Gene Silencing in Susceptible Citrus Leaves

1. Identification of Citrus Leaves Infected with Huanglongbing

Leaves of Lianjiang Citrus sinensis L. (hereinafter referred to as Citrus sinensis L.s) infected with citrus Huanglongbing were collected, and specific detection primers A2 and J5 were used to amplify a prlKAJL-rpoBC gene in the huanglongbing pathogen CLas for the identification of disease susceptibility of the citrus leaves (the target fragment length was 703 bp). At the same time, healthy Citrus sinensis L. leaves were used as a control.

The specific detection primer A2 for the Huanglongbing pathogen CLas:

(SEQ ID No. 13)
TATAAAGGTTGACCTTTCGAGTTT;

and
the specific detection primer J5 for the Huanglongbing pathogen CLas:

(SEQ ID NO. 14)
ACAAAAGCAGAAATAGCACGAACAA.

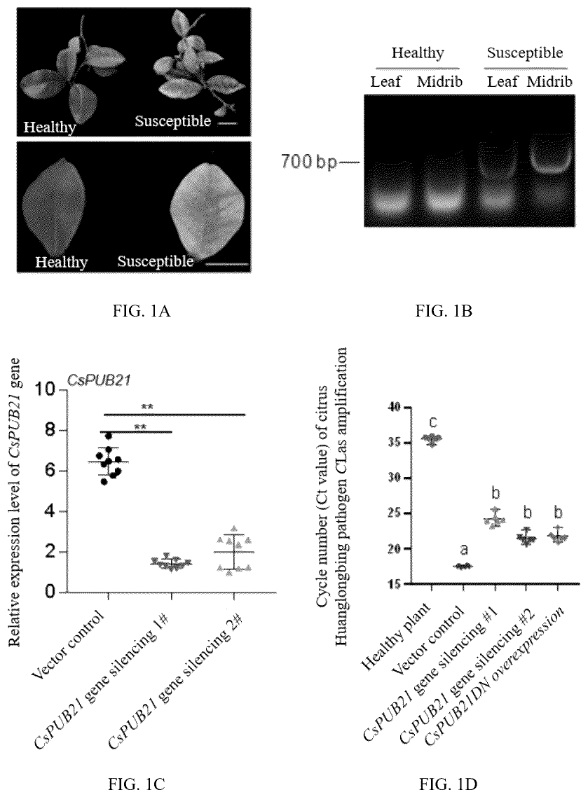

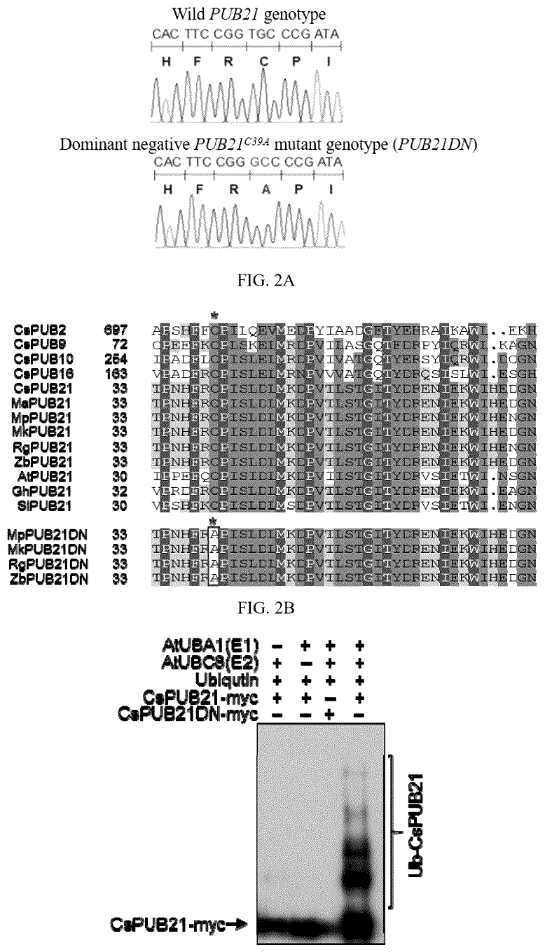

The results, as shown in FIG. 1A and FIG. 1B, show that the infected Citrus sinensis L. leaves are yellowed, and amplification to a fragment of about 700 bp in size can be achieved in the citrus leaves and midribs with an abundant bacterial load, whereas the fragment cannot be detected out in the healthy Citrus sinensis L. leaves and midribs thereof. Therefore, the susceptibility of the Citrus sinensis L. leaves is determined.

2. First, an LB plate (without antibiotics) was coated with Xanthomonascitrisub sp. Citri (Xcc), and incubated overnight at 28° C., bacteria were collected with sterile water, the OD_{600 nm} value was adjusted to be 0.5, then the Xcc bacterial solution (which weakened the immunity of citrus, and improved the expression of vector genes in citrus leaves) was injected with a 1 mL syringe into the Citrus sinensis L. leaves infected with Huanglongbing identified in step 1, to obtain the susceptible Citrus sinensis L. leaves injected with the Xcc bacterial solution, and the leaves continued to be put into a greenhouse for cultivation.

3. The RNAi gene silencing vectors CsPUB21 RNAi-1 and CsPUB21 RNAi-2 prepared in 4 of step I and the control vector (a vector 35S:YFP with a 35S promoter driving the expression of a YFP gene) were transformed into an Agrobacterium strain GV3101 by electroshocking, then the OD_{600 nm} value of the bacterial solution containing the above vectors was adjusted to 1.0, and the bacterial solution was left to stand at a room temperature for three hours or more.

4. 6-8 hours after the Xcc bacterial solution was injected into the leaves, the bacterial solution containing the gene silencing vectors and the control vector were separately infiltrated into the susceptible Citrus sinensis L. leaves injected with the Xcc bacterial solution by vacuum injection, and then the leaves were placed in a plant growth chamber for growth.

### III. The Pathogen Concentration May be Reduced by Reducing CsPUB21 Expression in the Susceptible Citrus Leaves

1. Leaf samples were collected after 3 days of growth, the RNA of the samples was extracted with an RNA extraction kit, cDNA was synthesized by reverse transcription, the CsPUB21 gene was tested using a fluorescence quantitative PCR, with COX as a reference gene, and the sequences of quantitative primers were as follows:
Citrus CsPUB21 gene detection primer CsPUB21-qF:

(SEQ ID No. 15)
CGTTGGGTCGTCGTCTATCGT;

Citrus CsPUB21 gene detection primer CsPUB21-qR:

(SEQ ID No. 16)
AATGGAGACTGCGAACTCCG;

Citrus endogamous gene detection primer COX-qF:

(SEQ ID No. 17)
GTATGCCACGTCGCATTCCAGA;

and
Citrus endogamous gene detection primer COX-qR:

(SEQ ID NO. 18)
GCCAAAACTGCTAAGGGCATTC.

The results, as shown in FIG. 1C, show that the CsPUB21 gene expression in the nine sample leaves silenced by CsPUB21 RNAi-1 and CsPUB21 RNAi-2 was significantly lower than that in the control leaves, which indicates that the CsPUB21 expression in these sample leaves was successfully reduced.

2. Leaf DNA was extracted by a CTAB method after 3 days of growth, the concentration of the Huanglongbing pathogen CLas in the susceptible leaves was detected by the fluorescence quantitative PCR, with COX as the reference gene, and the sequences of quantitative primers were as follows:
Huanglongbing pathogen CLas specific detection primer HLBas:

(SEQ ID No. 19)
GTCGAGCGCGTATGCAATACG;

Huanglongbing pathogen CLas specific detection primer HLBr:

(SEQ ID No. 20)
GCGTTATCCCGTAGAAAAAGGTAG;

Citrus reference gene detection primer COXf: GGTATGCCACGTCGCATTCCAGA (SEQ ID No.21); and
Citrus reference gene detection primer COXr: GCCAAAACTGCTAAGGGCATTC (SEQ ID No.22)

The results, as shown in FIG. 1D, show that both CsPUB21 RNAi-1 and CsPUB21 RNAi-2 are able to significantly reduce the concentration of CLas in the susceptible leaves, where CsPUB21 RNAi-2 is more effective and is able to reduce the number of pathogens by about 70%.

### Embodiment 2: Dominant Negative Mutant CsPUB21DN has CLas Resistance

### I. a Dominant Negative Mutant PUB21DN is Naturally Present in Rutaceae Plants

If PUB21 is a Huanglongbing (HLB) susceptible gene, there should be some differences between HLB-sensitive varieties and HLB-resistant varieties. To verify this, a PUB21 gene was cloned from Citrus sinensis, (sensitive), Murrayapaniculata (partially resistant), and Murrayakoenigii (fully resistant) using the same pair of full-length CsPUB21 amplification primers as in Embodiment 1.

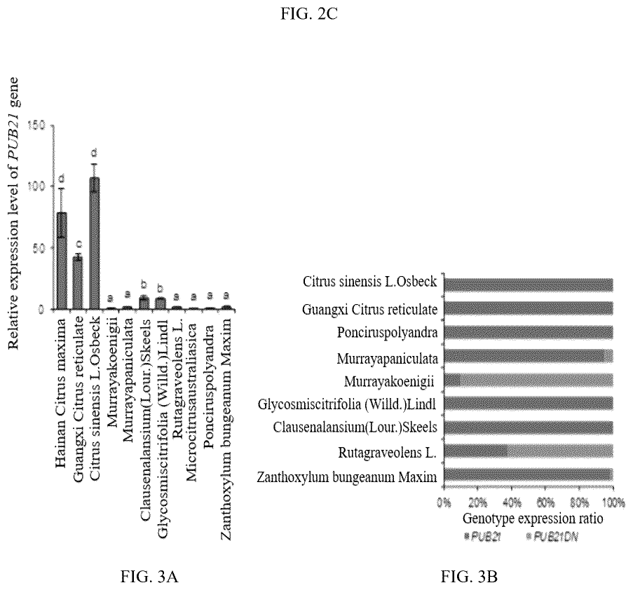

The results, as shown in FIG. 2A and FIG. 2B, show that gene sequencing indicates the presence of two PUB21 genotypes in Murrayapaniculata and Murrayakoenigii. One is wild-type PUB21, and the other has a point mutation (Cys-39 mutated to Ala, CsPUB21DN) in a conserved amino acid of a U-box structural domain, which is a dominant negative mutant of the known PUB21 gene and may impair the E3 ubiquitin ligase activity in related PUB, whereas Citrus sinensis only has wild-type PUB21.

To further validate this, the present disclosure also cloned a PUB21 gene from other HLB-resistant rutaceae relatives, such as Ponciruspolyandra, Microcitrusaustraliasica, Clausenalansium (Lour.) Skeels, Glycosmiscitrifolia (Willd.) Lindl, Zanthoxylum bungeanum Maxim and Rutagraveolens L.

The results show that Zanthoxylum bungeanum Maxim and Rutagraveolens L. have both PUB21 and PUB21DN genotypes, whereas only wild-type PUB21 is found in other HLB-resistant rutaceae relatives.

Based on clonal sequencing of the PUB21 gene and the NCBI database, the naturally present dominant negative mutant PUB21DN was not found in HLB-sensitive species and other non-rutaceae species.

### II. CsPUB21DN Blocks the E3 Ubiquitin Ligase Activity of CsPUB21

### 1. Vector Construction

First, vectors for in vitro ubiquitination detection in Escherichia coli were constructed, and full-length clones of CsPUB21 and CsPUB21DN (CsPUB21-C39A) were constructed into a pACYCDuet-CDS-Myc-AtUBC8-S vector by double digestion and ligation, respectively, to obtain vectors pACYCDuet-CsPUB21-Myc-AtUBC8-S and pACYCDuet-CsPUB21DN-Myc-AtUBC8-S, respectively.

The primer sequences for constructing wild-type vectors were as follows:

CsPUB21-Fw (BamHI):
(SEQ ID No. 23)
CAAGGGATCCATGATTTTGTCATGGAAAAGAC;
and
CsPUB21-Rv (StuI):
(SEQ ID No. 24)
CAAGAGGCCTAAACGGCCTTTTCAGGTCCT.
The primer sequences for constructing mutants
were as follows:
CsPUB21-C39A-Fw:
(SEQ ID No. 25)
CCGAACCACTTCCGGGCCCCGATATCTCTCGAC;
and
CsPUB21-C39A-RV:
(SEQ ID No.26)
TCTTTCATCAAGTCGAGAGATATCGGGGCC.

2. Construction of Escherichia coli Ubiquitination Detection System

pACYCDuet-CsPUB21/CsPUB21DN-Myc-AtUBC8-S, pCDFDuet-AtUBA1-S and pET-28a-FLAG-UBQ were co-transfected into BL21(DE3) competence, where CsPUB21 acted as E3 ubiquitin ligase, AtUBC8 acted as an E2 ubiquitin conjugating enzyme, AtUBA1 acted as an E1 ubiquitin activating enzyme, and UBQ acted as a ubiquitin small molecular protein Ubiquitin.

### 3. Analysis of CsPUB21 Auto-Ubiquitination Level

A strain containing an expression vector was cultured at 37° C. to OD_{600 nm} of 0.4-0.6, and after the addition of 500 nM of isopropyl β-D-thiogalactopyranoside (IPTG), a protein induced expression was carried out at 28° C. for 10 hours, followed by an overnight reaction at 4° C. A crude extracted protein substance was separated by 8% SDS-PAGE gel, and the CsPUB21 auto-ubiquitination level was analyzed by immunohybridization using an anti-c-myc tag antibody.

The results, as shown in FIG. 2C, show that in the reaction, no E3 ligase activity of PUB21 is observed in the absence of E1 or E2, but in the presence of both E1 (AtUBA1) and E2 (AtUBC8), it is shown that CsPUB21 can be polyubiquitinated and has the E3 ubiquitin ligase activity. The U-box structural domain is essential for the E3 ligase activity of PUB21, and CsPUB21DN does not show ubiquitination activity when highly conserved Cys-39 of the U-box structural domain in PUB21DN is mutated to Ala, which indicates that Cys-39 is essential for the maintenance of the enzymatic activity of PUB21.

### III. Reduction of CLas Titer in Susceptible Leaves by CsPUB21DN Expression

1. With the vector pENTR-3C-CsPUB21 obtained in step I of Embodiment 1 as a template, PCR amplification was carried out using mutant primers CsPUB21-C39A (CsPUB21-C39A-Fw and CsPUB21-C39A-Rv) to obtain an intermediate vector pENTR-3C-CsPUB21DN, and then the intermediate vector pENTR-3C-CsPUB21DN was ligated to the expression vector pH7-YFP-DC by the LR reaction using recombinase to obtain recombinant plasmid 35S:YFP-CsPUB21DN.

2. In accordance with the method in step II of Embodiment 1, the Huanglongbing susceptible leaves were first injected with the Xcc bacterial solution to obtain the susceptible Citrus sinensis L. leaves injected with the Xcc bacterial solution. The prepared 35S:YFP-CsPUB21DN and 35S:YFP control vectors were transformed into an Agrobacterium strain GV3101 by electroshocking, then the OD_{600 nm} value of the bacterial solution containing the above vectors was adjusted to 1.0, and the bacterial solution was left to stand at a room temperature for three hours or more. 6-8 hours after the Xcc bacterial solution was injected into the leaves, the bacterial solution with the OD_{600 nm} value of 1.0 was injected into the susceptible Citrus sinensis L. leaves injected with the Xcc bacterial solution by evacuation injection, and then the leaves were placed in the plant growth chamber for cultivation.

3. Leaf samples were collected after 3 days of growth, the leaf DNA was extracted by a CTAB method, and the concentration of the pathogen CLas in the midrib was detected by the fluorescence quantitative PCR. The larger the Ct value, the less the bacteria. The smaller the Ct value, the more the bacteria.

The results, as shown in FIG. 1D, show that the CLas concentration in the susceptible leaves is significantly reduced by CsPUB21DN overexpression.

### IV. Analysis of Expression Levels of PUB21 and PUB21DN Genes in Different Rutaceae Species

Samples from different Rutaceae species were collected, the RNA of the samples was extracted with a Plant RNA Mini Kit, the cDNA was synthesized by reverse transcription, and then the expression amount of the PUB21 and PUB21DN genes was detected by the fluorescence quantitative PCR. Primer sequences were as follows:

PUB21DN-qF:
(SEQ ID No. 27)
CCGGGGGTAGAAAGCGGGGG;
PUB21DN-qR:
(SEQ ID No. 28)
ACAATTGATCTCGAAAACCT;
PUB21-qF:
(SEQ ID No. 29)
TCGGGGGAAGAAAGCGGGAG;
and
PUB21-qR:
(SEQ ID NO. 30)
ACAATTGATCTCGAAAACCT.

The results, as shown in FIG. 3, show that the mRNA levels of PUB21 transcripts in HLB-resistant rutaceae relatives were significantly lower (FIG. 3A) compared to HLB-sensitive Citrus sp. species. The natural expression proportion of PUB21DN of the two PUB21 genotypes varied by species, where the expression proportion of PUB21DN in Murrayakoenigii or Rutagraveolens L. is greater than 60%, and the expression proportion of PUB21DN in Murrayapaniculata or Zanthoxylum bungeanum Maxim is less than 60% (FIG. 3B). In combination with field evaluation of HLB, transcript levels of PUB21, and the proportion of natural PUB21DN in different rutaceae species, the role of PUB21DN in HLB resistance is further confirmed.

### Embodiment 3: Prime Editing of CsPUB21 Gene to Mutate Position C39 to A39 (CsPUB21DN) can Effectively Resist Huanglongbing Pathogen CLas

In order to further confirm that CsPUB21DN can enhance the resistance function of citrus against the Huanglongbing pathogen CLas, a codon coding cysteine at position 39 of the CsPUB21 protein was site-specific edited by using a prime editor (Plant prime editing system (PE2(V2))) in citrus protoplast, and the codon was replaced from TGC to GCC, so mutation of an amino acid at position 39 of the CsPUB21 protein from Cys to Ala was realized artificially. The specific steps are as follows:

### I. Construction of a Prime Editing Vector PE2(Cs)-Peg-CsPUB21

1. Promoter amplification primers are designed, the primer sequences being as follows:

CsU6-Fw:
(SEQ ID No.31)
CGACGGCCAGTGCCAAGCTTGCGCTCAGGAGCCGGTTGAA;
CsU6-Rv:
(SEQ ID No. 32)
GTGTTGGTCTCGCAACCAACCTGGGAGTCCT;
CmYLCV-Fw:
(SEQ ID No. 33)
ACTGCTTGCTGCTTGGCAGACATACTGTCCCAC;
and
CmYLCV-Rv:
(SEQ ID No. 34)
TCCTAGGGAAGCTTAGCTCTTACCTGTTTTCGT,

where the nucleotide sequences shown underlined are homologous arm sequences at both ends of an original promoter of the prime editing vector PE2(V2).

With the cDNA of Citrus sinensis L.Osbeck as a template, PCR amplification was carried out using primers CsU6-Fw and CsU6-Rv to obtain a CsU6 promoter sequence containing the homologous arm and having a size of 665 bp, and the nucleotide sequence thereof was as shown in SEQ ID No. 35.

Tsingke Biotech was commissioned to synthesize the CmYLCV promoter sequence containing a homologous arm and having a size of 465 bp as shown in SEQ ID No.36.

2. A PE2(V2) intermediate fragment amplification primer was designed, the primer sequences being as follows:

Gap-Fw:
(SEQ ID No. 37)
TCCCAGGTTGGTTGCGAGACCAACACAAGGT;
Gap-Rv:
(SEQ ID No. 38)
GTATGTCTGCCAAGCAGCAAGCAGTATCGATC;
Cas9(H840A)-Fw:
(SEQ ID No. 39)
AAGAGCTAAGCTTCCCTAGGATGGCCCCTAAGA;
and
Cas9(H840A)-Rv:
(SEQ ID No. 40)
GATCCTCCGCTCTCTTAAGCTTCTTCTTCTTCGCCT,

where the nucleotide sequences shown underlined are homologous arm sequences at both ends of an intermediate fragment of the prime editing vector PE2(V2).

With the prime editing vector PE2(V2) as a template, PCR amplification was carried out using primers Gap-Fw and Gap-Rv to obtain a Gap intermediate fragment sequence containing a homologous arm and having a size of 328 bp, and the nucleotide sequence thereof was shown in SEQ ID No. 41.

With the prime editing vector PE2(V2) as a template, PCR amplification was carried out using primers Cas9(H840A)-Fw and Cas9(H840A)-Rv to obtain a Cas9(H840A) intermediate fragment sequence containing a homologous arm and having a size of 5206 bp, and the nucleotide sequence thereof was shown in SEQ ID No. 42.

3. The PE2(V2) vector was singly digested with restriction endonuclease Hind III to obtain a backbone vector with a size of 11733 bp; then the CsU6 promoter gene with a size of 665 bp in step 1 and the Gap intermediate fragment sequence with a size of 328 bp in step 2 were sequentially subjected to homologous recombinant using C112 homologous recombinase; and the CmYLCV promoter gene with a size of 465 bp and a Cas9(H840A) intermediate fragment with a size of 5206 bp were subjected to homologous recombinant; then the product of the two recombinations was homologously recombined for the third time to obtain the recombination sequence with a size of 6646 bp; finally, the backbone vector was recombined with the recombination sequence with a size of 6646 bp for the last time; and after ligation and transformation, the promoter-optimized PE2(Cs)-CsU6 promoter-Gap-CmYLCV promoter-Cas9(H840A) vector (hereinafter referred to as PE2(Cs)) was obtained.

4. Tsingke Biotech was commissioned to synthesize the peg-CsPUB21-sgRNA gene containing a homologous arm and having a size of 169 bp as shown in SEQ ID No. 43.

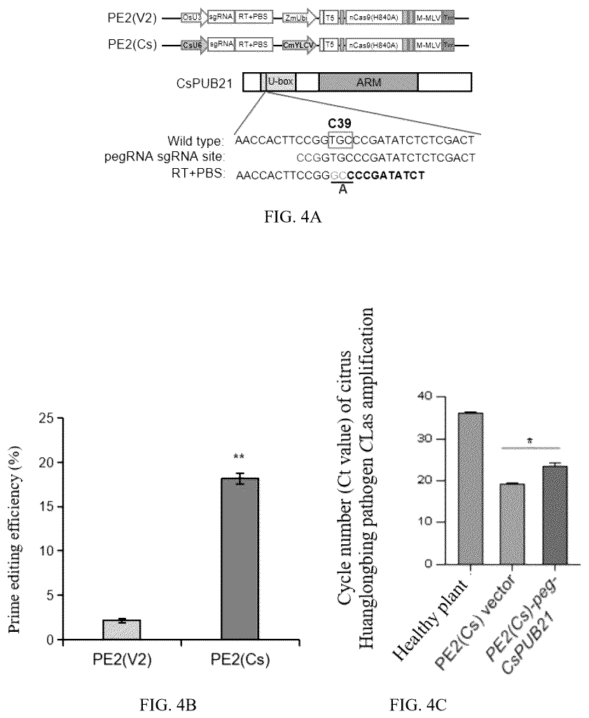

5. The PE2(Cs) vector was singly digested with restriction endonuclease BsaI to obtain a backbone vector; and then the backbone vector was ligated with the peg-CsPUB21-sgRNA gene fragment with a size of 169 bp in step 4 using the C112 homologous recombinase to obtain the PE2(Cs)-peg-CsPUB21 prime editing vector, where the vector construction process was as shown in FIG. 4A.

### II. Analysis of Editing Efficiency of the PE2(Cs)-Peg-CsPUB21 Prime Editing Vector in Citrus Protoplast

### 1. Mass Extraction of PE2(Cs)-Peg-CsPUB21 Recombinant Plasmid

The PE2(Cs)-peg-CsPUB21 prime editing vector was transferred into the DH5a competent cell and streaked on a solid LB plate containing Kan antibiotic overnight, single colonies were picked and shaken in 10 mL of liquid LB containing Kan antibiotic overnight, and the bacterial solution was transferred to 200 mL of liquid LB containing Kan antibiotic and shaken for 12 hours, and the bacteria were collected for plasmid extraction using Tiangen (endotoxin-free) plasmid extraction kit. At the same time, the PE2(Cs) vector was used as a control.

2. The extracted PE2(Cs)-peg-CsPUB21 recombinant plasmid was used for the transformation of citrus protoplast. First, young Citrus sinensis L. leaves infected with citrus Huanglongbing were collected (identification of susceptibility to the disease was carried out in accordance with the method in 1 of step II of Embodiment 1), rib portions of the leaves were removed, and the leaves were cut into thin strips and immersed in a citrus leave enzyme digestion solution (0.7 M mannitol, 1M MES, 2.4% cellulose R10, 1.2% Macerozyme R10, 0.1% BSA, and 1 mM CaCl₂)) to be shaken in dark place for 5 h; an enzyme solution was filtered through a gauze, and after two washes, protoplast precipitate was collected and placed on ice for about 30 min; PEG4000-mediated protoplast was used for transformation of the PE2 (Cs)-peg-CsPUB21 recombinant plasmid in a water bath of 37° C., supernatant was removed by brief centrifugation after 30 min, and the protoplast was added into a protoplast culture medium W5 solution (154 mM NaCl, 125 mM CaCl₂), 5 mM KCl, 5 mM glucose, 0.03% MES, pH 5.8) and cultured for 72 h under dark conditions.

3. Samples were collected after the protoplast was cultured for 72 hours, the protoplast DNA was extracted using the CTAB method, a region where the prime editing sequence was located was amplified with specific primers (PUB21-qF and PUB21-qR), a PCR product was gelled and purified and ligated with the TA vector, Escherichia coli competent cells are transformed, 50 monoclones were randomly selected for sequencing, and the ratio of the number of mutant cells to the total number of sequenced cells was calculated to estimate the editing efficiency of the vector: vector editing efficiency (%)=(the number of mutated cells/50)×100.

The results, as shown in FIG. 4B, show that the codon TGC at the amino acid of position 39 of the key enzyme activity site of CsPUB21 can be effectively replaced with GCC by the prime editing technique in the citrus protoplast, and the editing efficiency of the optimized prime editing vector PE2(Cs) was increased from 2%, the original editing efficiency of the vector PE2(V2), to 18.2%, which indicates the effectiveness of the vector PE2(Cs).

### III. PE2(Cs)-Peg-CsPUB21 Prime Editing can Reduce CLas Titer in Susceptible Leaf Protoplast

The PE2(Cs)-peg-CsPUB21 plasmid was introduced into protoplast according to the methods described in 1 and 2 of step II, then the protoplast DNA was extracted using the CTAB method, and then the concentration of the Huanglongbing pathogen CLas in citrus protoplast after PE2(Cs)-peg-CsPUB21 editing was detected by the fluorescence quantitative PCR. At the same time, the PE2(Cs) vector was used as a control.

The results, as shown in FIG. 4C, show that the CLas concentration in the susceptible citrus protoplast after PE2(Cs)-peg-CsPUB21 editing is significantly reduced.

### Embodiment 4: CsPUB21 Enhances Attractiveness of Citrus to Diaphorina citri Kuwayama by Reducing Terpene Compounds

I. CsPUB21 Overexpression Increased the Attractiveness of Citrus to Diaphorina citri Kuwayama, while CsPUB21DN Overexpression had the Opposite Effect

### 1. Preparation of Transgenic Citrus Overexpressing CsPUB21 and Transgenic Citrus Overexpressing CsPUB21DN

In order to obtain stable transgenic citrus plants, CsPUB21 and CsPUB21DN full-length cDNAs were ligated into the pLGN135 vector (driven by the 35S promoter for expression) to obtain the recombinant vectors CsPUB21-pLGN135 and CsPUB21DN-pLGN135, respectively; the recombinant vectors CsPUB21-pLGN135 and CsPUB21DN-pLGN135 were introduced into the Agrobacterium strain EHA105 to obtain recombinant bacteria CsPUB21-pLGN135/EHA105 and CsPUB21DN-pLGN135/EHA105; the recombinant bacteria CsPUB21-pLGN135/EHA105 and CsPUB21DN-pLGN135/EHA105 were utilized; young leaf columns of Valencia oranges were used as materials for Agrobacterium-mediated transformation for genetic transformation, and then micrografted into Citrus aurantium seedlings, followed by further grafting of the resulting plants into Citrus aurantium main stalks in the greenhouse, to obtain CsPUB21 transgenic plants and CsPUB21DN transgenic plants, respectively, and the detailed transformation steps can be found in the literature “Hongwei Shi, Zuhui Yang, Jie Huang, Haodi Wu, Shimin Fu, Weimin Li, Xiuping Zou, Changyong Zhou, Xuefeng Wang. An effector of ‘Candidatus Liberibacter asiaticus’ manipulates autophagy to promote bacterial infection. J Exp Bot, 2023, 74(15):4670-4684”; the primers CsPUB21-qF and CsPUB21-qR were used to detect the expression of CsPUB21 and CsPUB21DN genes in the transgenic plants by qPCR, respectively, the CsPUB21DN transgenic plants also needed to be sequenced to determine the presence of the mutant site, so as to obtain the transgenic citrus overexpressing CsPUB21 and transgenic citrus overexpressing CsPUB21DN (CsPUB21DN overexpression 1 # and CsPUB21DN overexpression 2 #). At the same time, the transgenic citrus transfected with a pLGN135 vector was used as a control (vector control).

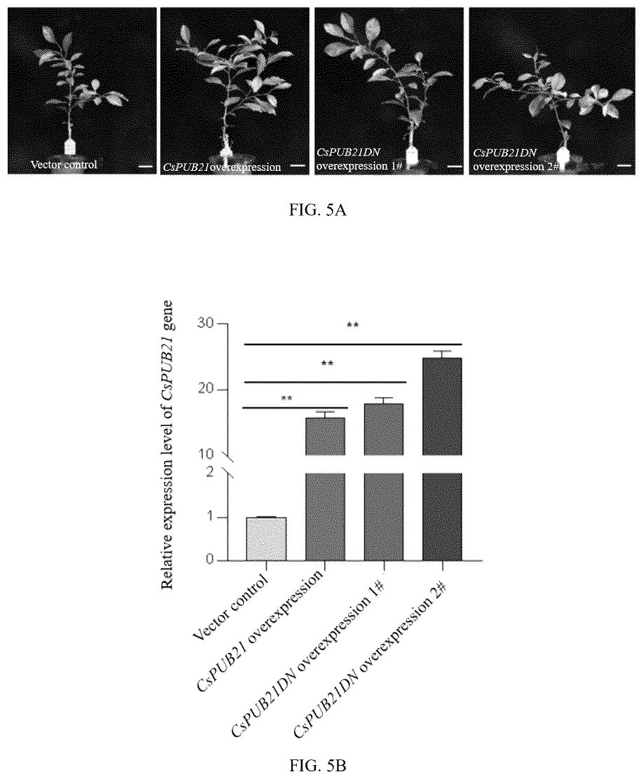

The results, as shown in FIG. 5A and FIG. 5B, show that the expression of CsPUB21 gene in the plant leaves of transgenic citrus overexpressing CsPUB21 and transgenic citrus overexpressing CsPUB21DN were significantly higher than that in the control leaves, indicating that the expression of CsPUB21 was significantly improved in the plants of transgenic citrus overexpressing CsPUB21 and transgenic citrus overexpressing CsPUB21DN.

2. Dual-Selection Experiment for Diaphorina citri Kuwayama

The plant leaves of transgenic citrus overexpressing CsPUB21 and transgenic citrus overexpressing CsPUB21DN were subjected to a double-selection experiment for Diaphorina citri Kuwayama: plant leaves of the transgenic citrus overexpressing CsPUB21 or CsPUB21DN of similar sizes were selected and placed in a selection petri dish (150 mm*25 mm) together with control leaves, 100 pieces of Diaphorina citri Kuwayama were caught for starvation treatment for 2 h and then placed in the selection petri dish for release, and the number of Diaphorina citri Kuwayama on each leaf was counted after 40 min.

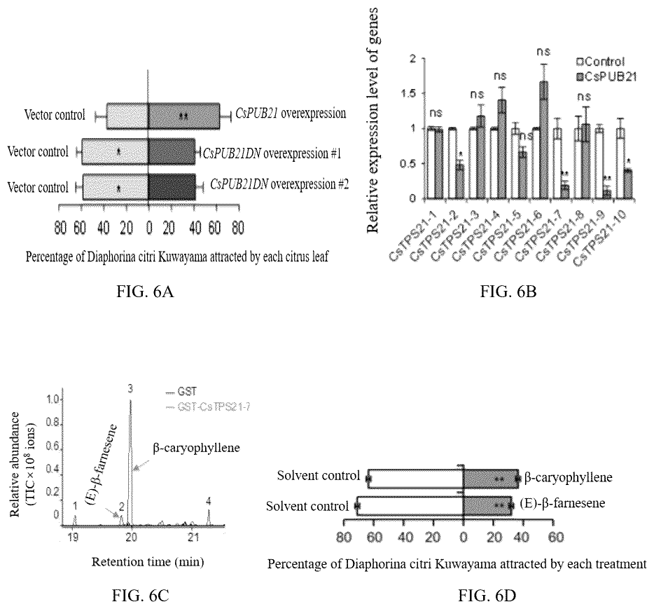

The results of the double-selection experiment for Diaphorina citri Kuwayama, as shown in FIG. 6A, show that the leaves of the transgenic citrus overexpressing CsPUB21 are able to attract 63.5% of Diaphorina citri Kuwayama, whereas the control leaves attract 36.5% of Diaphorina citri Kuwayama, thus indicating that the increased expression level of CsPUB21 in leaves increases the attraction to Diaphorina citri Kuwayama. The leaves of the transgenic citrus overexpressing CsPUB21DN are able to attract about 40% of Diaphorina citri Kuwayama, whereas the control leaves attract 60% of Diaphorina citri Kuwayama, indicating that CsPUB21DN exhibits an enhanced avoidance effect for Diaphorina citri Kuwayama after being overexpressed. It is reported that the host selection preference of citrus infected with Huanglongbing for Diaphorina citri Kuwayama may be increased, thus CsPUB21 negatively regulates the resistance of citrus to Diaphorina citri Kuwayama.

### II. CsPUB21 Expression Inhibits Synthesis of Terpene Compounds

The transgenic citrus leaves after Diaphorina citri Kuwayama double selection were collected, and the sample RNA was extracted with a Plant RNA Small Dose Extraction Kit, and cDNA was synthesized by reverse transcription, followed by the fluorescence quantitative PCR for the detection of terpene synthase CsTPSs genes. The sequences of detection primers are shown in Table 1.

TABLE 1

| | |
|---|---|
| CsTPS21-1 F | TGCAACAGTATGGCGTGTCT (SEQ ID No. 44) |
| CsTPS21-1 R | CCACCATCATCCGAGCAAGA (SEQ ID No. 45) |
| CsTPS21-2 F | TTGGCGTGGCCTATCACTTT (SEQ ID No. 46) |
| CsTPS21-2 R | ATGTTGCCGTAGAAGCCGAA (SEQ ID No. 47) |
| CsTPS21-3 F | GACCTTGGCAGCGATAGTGA (SEQ ID No. 48) |
| CsTPS21-3 R | GTGTGCTGCCTCGTACAAAC (SEQ ID No. 49) |
| CsTPS21-4 F | GGCAGCACACCTAGCCATTA (SEQ ID No. 50) |
| CsTPS21-4 R | TAGGGCTTTACGGAGTGGGA (SEQ ID No. 51) |
| CsTPS21-5 F | TCCTTGGCATGTGTGAGGTT (SEQ ID No. 52) |
| CsTPS21-5 R | AGCATTCAACTCCTGTGGTAAC (SEQ ID |
| | No. 53) |
| CsTPS21-6 F | TGGCAAAGAAAAGATGCAAGAGC (SEQ ID |
| | No. 54) |
| CsTPS21-6 R | TGATGACGCTGTTGAAATCTTAGGG (SEQ ID |
| | No. 55) |
| CsTPS21-7 F | GAAACCATTGGCAGGCTCAT (SEQ ID No. 56) |
| CsTPS21-7 R | CGTGCGAAATAAAGAAGCCGT (SEQ ID |
| | No. 57) |
| CsTPS21-8 F | TGACGAATGACGTTGAAGGCA (SEQ ID |
| | No. 58) |
| CsTPS21-8 R | TAACCTTGATACGGCTCTACGG (SEQ ID |
| | No. 59) |
| CsTPS21-9 F | AGTGGTTTCCATGACTTCTGCT (SEQ ID |
| | No. 60) |
| CsTPS21-9 R | AGGCAGTCTAATGCAACTGTCA (SEQ ID |
| | No. 61) |
| CsTPS21-10 F | GCAACCGTTTCCAGTTCAACA (SEQ ID |
| | No. 62) |
| CsTPS21-10 R | ATTGCAACTTCTGGGCAGGT (SEQ ID No. 63) |

Results are as shown in FIG. 6B, CsPUB21 overexpression significantly reduces the expression of four CsTPS21 genes (CsTPS21-2, CsTPS21-7, CsTPS21-9, and CsTPS21-10) compared with the control, indicating that CsPUB21 negatively regulates the expression of terpene synthase genes.

### III. The Main Terpene Product of CsTPS21-7 is β-Caryophyllene

### 1. Vector Construction

Primers CsTPS21-7-Fw (kpnI) and CsTPS21-7-Rv (EcoRI) were used to amplify the cDNA of the CsTPS21-7 gene from the transcript of Citrus sinensis L.Osbeck to obtain a PCR amplification product, which contained a CsTPS21-7 full-length gene with a length of 1557 bp; then the PCR amplification product was cloned into the pENTR-3C vector through restriction endonuclease KpnI and EcoRI; and finally the CsTPS21-7 full-length gene in the pENTR-3C vector was ligated into the pGEX-DC-3HA vector by homologous recombination to obtain a pGEX-CsTPS21-7-3HA vector. The primers for vector construction were as follows:

CsTPS21-7-Fw (kpnI):
(SEQ ID No. 64)
CAAGGGTACCATGAAAGATATGTCTATTCCA;
and
CsTPS21-7-Rv (EcoRI):
(SEQ ID No.65)
CAAGGAATTCATCGCAAGGGGTTCCGTCAG.

### 2. Protein Induction and Purification

The pGEX-CsTPS21-7-3HA vector constructed in step 1 was transferred into BL21(DE3) competence for protein induction and purification, and a GST-CsTPS21-7 fusion protein was obtained by purification through Glutathione Sepharose magnetic beads.

The pENTR-3C vector was transferred into BL21(DE3) competent cell for protein induction and purification, and a GST-tagged protein was obtained by purification through Glutathione Sepharose magnetic beads.

### 3. Functional Analysis of CsTPS21-7

2 μg of GST or GST-CsTPS21-7 fusion protein, 10 μg of (E,E)-farnesyl diphosphate (FPP) and reaction buffer were placed in a 2 mL PTFE sealed glass tube, and SPME fiber adsorbent needles were placed into the headspace of the tube, and reaction samples were cultivated at 30° C. for 30 min, and the adsorbent needles were transferred to GC-MS for detection of headspace volatiles.

Results are as shown in FIG. 6C, the major sesquiterpene product of CsTPS21-7 was initially identified as β-caryophyllene (accounting for 81.81±0.92% of the total product), and the other three minor products are 7-epi-sesquiterpene, (E)-β-farnesene, and sesquiterpene, which account for 4.79±0.11%, 5.54±0.32%, and 7.86±0.70% of the total compounds, respectively.

IV. β-Caryophyllene has Diaphorina citri Kuwayama Avoidance and Fungicidal Effects

In order to determine the effects of the main terpene product of CsTPS21-7 on Diaphorina citri Kuwayama, a double-selection experiment for Diaphorina citri Kuwayama was carried out. The specific experimental steps were as follows: firstly, 100 μg of β-caryophyllene and (E)-β-farnesene were dissolved in 100 μL of n-hexane to obtain a β-caryophyllene solution and an (E)-β-farnesene solution at a concentration of 1 μg/L, respectively, and the β-caryophyllene and (E)-β-farnesene solution solvent at a concentration of 1 μg/L was applied to citrus leaves, and the solvent n-hexane was used as the negative control. Similarly sized β-caryophyllene-treated leaves or (E)-β-farnesene-treated leaves were selected to be placed in a 150 mm*25 mm selection petri dish together with the control leaves, 100 pieces of Diaphorina citri Kuwayama were captured for starvation treatment for 2 h, and then placed in the selection petri dish to be released, and the number of Diaphorina citri Kuwayama on each leaf was counted after 40 min.

The results, as shown in FIG. 6D, show that β-caryophyllene and (E)-β-farnesene have a significant repulsive effect on Diaphorina citri Kuwayama compared to the solvent n-hexane. In addition, it has been shown that β-caryophyllene is the major compound of Murrayapaniculata plants, accounts for 57.57% of terpene products of Murrayapaniculata, and has a rapid bacterial and fungal killing effect. Therefore, these results suggest that CsPUB21 promotes the replication and spread of citrus Huanglongbing by inhibiting the synthesis of β-caryophyllene.

### Embodiment 5: Citrus Huanglongbing Resistance-Related Protein MYC2 is a Directly Acting Substrate for PUB21

### I. Yeast Two-Hybrid Screening to Identify the Presence of the Interaction Between MYC2 and PUB21

### 1. Vector Construction

The vector pENTR-3C-CsPUB21 obtained in step I of Embodiment 1 was ligated using recombinase and the expression vector pGBT9 by the LR reaction to obtain a recombinant vector BD-CsPUB21.

The vector pENTR-3C-CsPUB21DN obtained in step III of Embodiment 2 was ligated using recombinase and the expression vector pGBT9 by the LR reaction to obtain a recombinant vector BD-CsPUB21DN.

At the same time, according to the structural domain of the CsPUB21 protein, CsPUB21 truncated yeast expression vectors BD-CsPUB21^{U-box} (the expressed CsPUB21 truncated CsPUB21^{U-box} is positions 1-134 of the amino acid sequence of the CsPUB21 protein), and BD-CsPUB21^{ARM} (the expressed CsPUB21 truncated CsPUB21^{ARM} is positions 135-444 of the amino acid sequence of the CsPUB21 protein) were constructed.

2. Yeast two-hybrid library screening was performed using BD-CsPUB21 as bait to obtain several potential interaction targets, including CsMYC2.

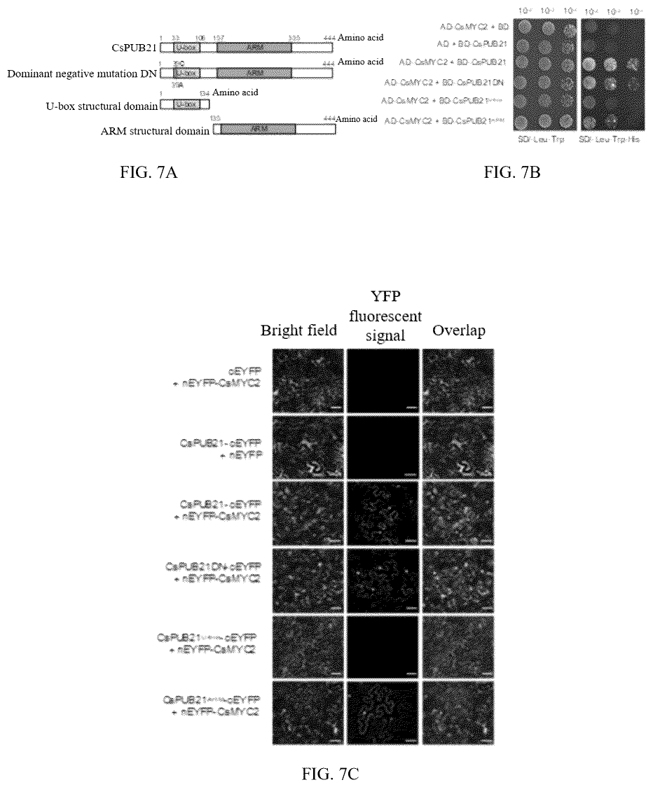

3. To confirm the interaction between CsPUB21 and CsMYC2, citrus CsMYC2 was full-length cloned into the pGAD424 vector by the LR reaction to generate AD-CsMYC2, and a yeast strain AH109 was subsequently co-transformed with an AD fusion structure and a BD fusion structure. The yeast co-transformed according to the plasmid combinations shown in FIG. 7B was placed on a synthetic medium SD-Leu-Trp-His containing 5 mM 3-amino-1,2,4-triazole (3-AT). Blank vectors pGBKT7 (BD) and pGADT7 (AD) were used as negative controls.

The results, as shown in FIG. 7B, show that the presence of the interaction between CsPUB21 and CsMYC2 was confirmed by yeast two-hybridization, CsPUB21DN does not affect the interaction with CsMYC2, and the ARM structural domain of CsPUB21 was able to have full-length interaction with CsMYC2.

II. MYC2 and PUB21 have an Interaction Relationship in Plants

1. The vector pENTR-3C-CsPUB21 obtained in step I of Embodiment 1 was cloned into the fluorescent complementary expression vector pSAT1-cEYFP using recombinase by the LR reaction to obtain CsPUB21-cEYFP.

The vector pENTR-3C-CsPUB21DN obtained in Step III of Embodiment 2 was cloned into the fluorescent complementary expression vector pSAT1-cEYFP using recombinase by the LR reaction to obtain CsPUB21DN-cEYFP.

At the same time, CsPUB21 truncated expression vectors CsPUB21^{U-box}-cEYFP and CsPUB21^{ARM}-cEYFP were constructed according to the structural domain of the CsPUB21 protein.

pENTR-3C-CsMYC2 was cloned into a fluorescent complementary expression vector pSAT1-nEYFP using recombinase by the LR reaction to obtain nEYFP-CsMYC2.

2. The vector constructed in step I was transformed into the Agrobacterium EHA105 strain by electroshock transformation, recombinant Agrobacterium was collected, bacteria were resuspended with an equal volume of MMA (10 mM MgCl₂, 10 mM MES, 150 μM Acetosyringone) liquid, the OD_{600 nm} value was adjusted to 1.5, and the bacteria were left to stand at a room temperature for three hours or more.

3. The vector fused with cEYFP and the vector liquid fused with nEYFP were evenly mixed according to the volume ratio of 1:1, injected into tobacco leaves with a 1 ml syringe, and then placed in the greenhouse for cultivation.

4. After 2-3 days, a fluorescence signal of EYFP was recorded by an SP8 confocal microscope.

The results, as shown in FIG. 7C, show that the interaction of CsPUB21 and CsMYC2 in the cytoplasm and nucleus was confirmed by BiFC analysis, and the dominant-negative mutant PUB21DN does not affect the interaction with CsMYC2.

### Embodiment 6: Direct Ubiquitination and Degradation of CsMYC2 with Huanglongbing Bacteria Resistance by CsPUB21

### I. CsPUB21 Directly Ubiquitinates CsMYC2

1. The full length of a CsMYC2 gene was ligated into the pCDFDuet-MBP-DC-HA-AtUBA1-S vector by double digestion of EcoRI and StuI to obtain a pCDFDuet-MBP-CsMYC2-HA-AtUBA1-S vector. The primer sequences of the constructed vectors were as follows:

CsMYC2-Fw (EcoRI):
(SEQ ID No. 66)
CAAGGAATTCATGACGGACTACCGGTTACC;
and
CsMYC2-Rv (StuI):
(SEQ ID No. 67)
CAAGAGGCCTTTGGGTATCTCCAACTTTGG.

2. The pACYCDuet-CsPUB21/CsPUB21DN-Myc-AtUBC8-S vector obtained in step II of Embodiment 2, and the pCDFDuet-MBP-CsMYC2-HA-AtUBA1-S vector and the pET-28a-FLAG-UBQ vector constructed in step I were co-transfected into a BL21(DE3) competent cell. The strains containing the expression vectors were cultured and induced, crude extracted proteins were separated by 8% SDS-PAGE gel, and immunohybridization analysis of the levels of CsPUB21 auto-ubiquitination and substrate CsMYC2 ubiquitination was carried out using anti-c-myc and anti-MBP tag antibodies.

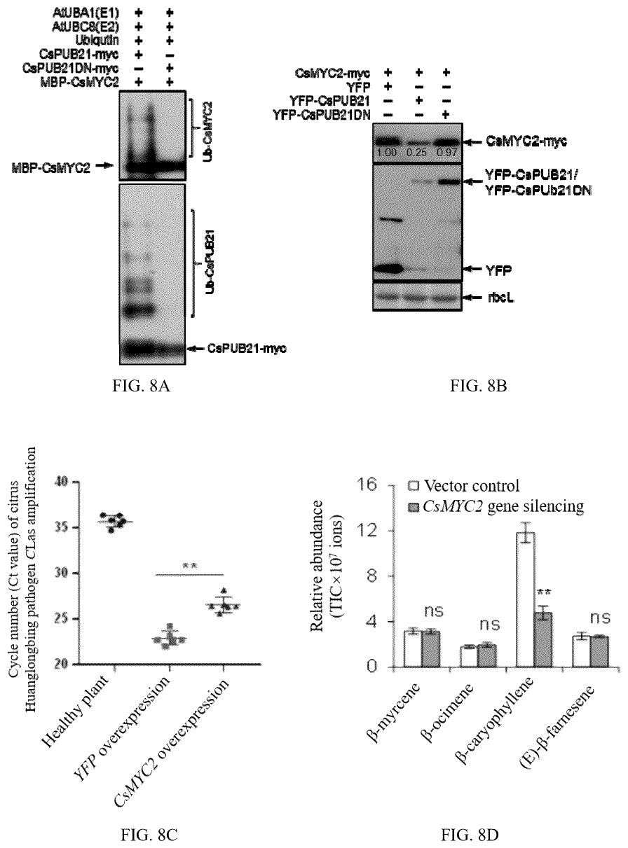

The results, as shown in FIG. 8A, show that CsPUB21 is able to effectively polyubiquitinate the CsMYC2 protein in vitro, but CsPUB21DN cannot ubiquitinate CsMYC2.

### II. CsPUB21 was Able to Degrade CsMYC2 in Plants

1. pENTR-3C-CsMYC2 was cloned into the pBA-DC-myc vector by the LR reaction using recombinase, and a 35S:CsMYC2-myc vector was constructed.

2. The 35S:CsMYC2-myc vector, the 35S:YFP-CsPUB21 vector obtained in Embodiment 1, the 35S:YFP-CsPUB21DN vector obtained in step III of Embodiment 2, and the control vector 35S:YFP were transformed into the Agrobacterium tumefaciens strain GV3101 by electroshock transformation, respectively, recombinant Agrobacterium was collected, bacteria were resuspended with an equal volume of MMA liquid, the OD_{600 nm} value was adjusted to 1.5, and the bacteria were left to stand at room temperature for three hours or more.

3. The bacterial solution containing the 35S:CsMYC2-myc expression vector was evenly mixed with the bacterial solution containing the 35S:YFP-CsPUB21 vector, the 35S:YFP-CsPUB21DN vector, or the 35S:YFP vector, respectively, in a volume ratio of 1:1, injected into tobacco leaves with a 1 ml syringe, and then placed in the greenhouse for cultivation.

4. After 2-3 days, the injected expressed tobacco leaf samples were collected, the extracted total proteins were electrophoresed by 10% SDS-PAGE gel, and then immunohybridization analysis was carried out using anti-c-myc and anti-YFP tag antibodies.

The results, as shown in FIG. 8B, show that the accumulated level of the CsMYC2 protein and coexpression of CsPUB21 were significantly reduced, but this reduction was inhibited by coexpression of CsPUB21DN, which indicates that MYC2 is a direct ubiquitination substrate for PUB21.

### III. Overexpression of CsMYC2 to Reduce the Concentration of the Pathogen CLas in Susceptible Citrus Leaves

CsMYC2 was overexpressed in the susceptible citrus leaves according to the method in step II of Embodiment 1. Leaf samples were collected after 3 days of growth, and leaf DNA was extracted by the CTAB method, and the concentration of the Huanglongbing pathogen CLas in the midribs of the leaves was detected by the fluorescence quantitative PCR.

The results, as shown in FIG. 8C, show that CsMYC2 overexpression significantly reduces the concentration of CLas in the susceptible leaves, which indicates that MYC2 has an anti-Huanglongbing effect.

### IV. CsMYC2 Positively Regulates Terpene Chemosynthesis

1. Silencing of CsMYC2 in healthy citrus: CsMYC2 gene silencing in healthy citrus was carried out according to the MYC2 gene silencing method described in step IV of Embodiment 2 in the invention patent application document No. CN115160421A to obtain CsMYC2 gene-silenced citrus (denoted as CsMYC2VIGS).

2. Detection of headspace volatiles in CsMYC2VIGS plants: Headspace volatiles of the CsMYC2 gene-silenced citrus were collected by solid-phase microextraction, and analyzed and identified by gas chromatography-mass spectrometry (GC-MS).

The results, as shown in FIG. 8D show that compared with control citrus, the emission level of β-caryophyllene, the main product of CsTPS21-7, was only 40.2% in the CsMYC2 gene-silenced citrus. These results indicate that MYC2 positively regulates the defense response of plants against citrus huanglongbing, and that MYC2 is an anti-citrus huanglongbing disease-related protein.

### Embodiment 7: Transgenic Citrus Overexpressing CsPUB21DN can Effectively Reduce the Infestation of Huanglongbing Bacteria

### 1. Grafting for Bacteria Infection

Branches of Gannan navel oranges infected with citrus Huanglongbing in Datian, Gannan (identification of disease susceptibility was carried out according to the method in 1 of step II of Embodiment 1) were grafted into the vector control and transgenic citrus overexpressing CsPUB21 and CsPUB21DN obtained in step I of Embodiment 4, and continued to be cultivated in a greenhouse, and after 2 and 3 months, the titer of the Huanglongbing pathogen CLas in newborn leaves of transgenic citrus was detected by qPCR according to the method in step III of Embodiment 1, respectively.

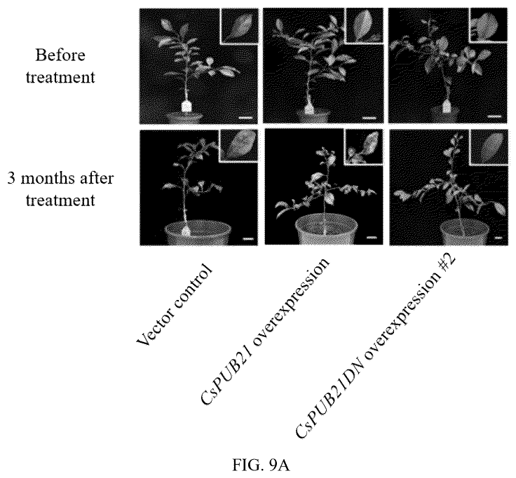

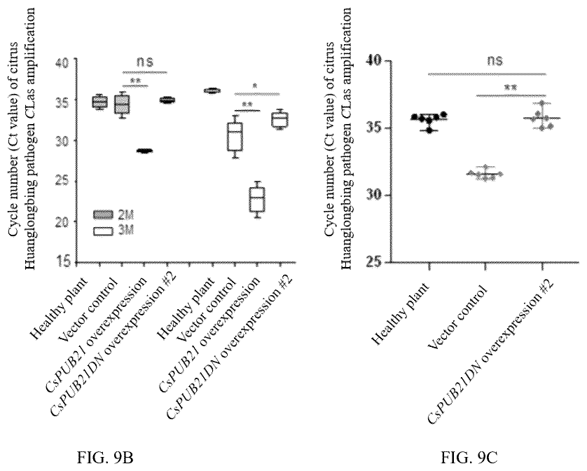

The results show that 3 months after healthy transgenic plants re-grafted with bacterial citrus branches using grafting for bacteria infection, it is observed that both vector control and transgenic citrus plants overexpressing CsPUB21 show disease characteristics of Huanglongbing infection, with yellowing and crumpling of leaves, but the transgenic citrus plants overexpressing CsPUB21DN still show a healthy state (FIG. 9A). The detection of the titer of the Huanglongbing pathogen Clas shows that at 2 and 3 months of pathogen infestation, the pathogen titer of the transgenic citrus plants overexpressing CsPUB21 is significantly higher than that of the vector control, whereas at 3 months of infestation, the plants overexpressing CsPUB21DN can be detected out to have the pathogen, but with lower pathogen titer than the vector control plants (FIG. 9B).

2. Bacterium Infection by Feeding of Bacteria-Carrying Diaphorina citri Kuwayama

Under natural environmental conditions, Huanglongbing pathogens are often transmitted through feeding of Diaphorina citri Kuwayama. Therefore, in order to further test the insect resistance of the transgenic citrus plants overexpressing CsPUB21DN, Diaphorina citri Kuwayama (identification of Diaphorina citri Kuwayama carrying the CLas was carried out according to the method in 1 of step II of Embodiment 1) carrying the Huanglongbing pathogen CLas obtained from Datian, Gannan were placed, by bacterial transmission via Diaphorina citri Kuwayama, on the branches of the vector control and the transgenic citrus overexpressing CsPUB21DN obtained in step I of Embodiment 4, respectively, for natural bacterial inoculation by feeding of Diaphorina citri Kuwayama, and after 7 days, the titer of the Huanglongbing pathogen CLas in the transgenic citrus leaves was detected by qPCR according to the method in step III of Embodiment 1.

The results show that there was no difference in the Ct values detected between the transgenic citrus plants overexpressing CsPUB21DN and the healthy plants, which is significantly lower than that of the vector control plants (FIG. 9C), which indicates that overexpression of CsPUB21DN in citrus plants is able to improve the resistance effect of citrus to Huanglongbing infestation.

The present disclosure is described in detail above. For those skilled in the art, the present disclosure can be practiced in a wide range of equivalent parameters, concentrations and conditions without departing from the purpose and scope of the present disclosure and without unnecessary experimentation. Although particular embodiments of the present disclosure are given, it should be understood that further improvements can be made to the present disclosure. In summary, in accordance with the principles of the present disclosure, the present application is intended to encompass any changes, uses or improvements to the present disclosure, including changes out of the scope of what has been disclosed in the present application and made with conventional techniques known in the art. The application of some of the essential features may be carried out according to the scope of the following appended claims.

### INDUSTRIAL APPLICABILITY

The present disclosure identifies a citrus susceptibility factor CsPUB21, which can promote the incidence of citrus Huanglongbing, and a mechanism of a dominant negative mutant thereof CsPUB21DN to enhance disease resistance, and provides a gene resource for effectively resisting the replication and spread of Huanglongbing pathogens. In practical applications, the plant resistance of the plant to Huanglongbing and a spread vector thereof, i.e. Asian Diaphorina citri Kuwayama, can be regulated and controlled by regulating and controlling the content and/or activity of the CsPUB21 or CsPUB21DN protein of the plant, so that Huanglongbing-resistant plant varieties are cultivated. The present disclosure is important for the prevention and control of Huanglongbing in plants.

1. Application of a PUB21 protein or a biomaterial related thereto in any one of A1)-A3):
A1) regulation and controlling of plant resistance to Huanglongbing;
A2) regulation and controlling of insect resistance of a plant; or
A3) plant breeding, wherein
the PUB21 protein is any one of a1)-a4):
a1) a protein with an amino acid sequence of SEQ ID No. 1;
a2) a fused protein obtained by ligating a protein-tag at an N-terminus and/or a C-terminus of the amino acid sequence in a1);
a3) a protein with similar function obtained by subjecting the amino acid sequence in a1) to substitution and/or deletion and/or addition of one or several amino acid residues; or
a4) a protein having 75% or more identity to the amino acid sequence in a1) and having similar function; and
the biomaterial is a nucleic acid molecule coding the PUB21 protein or an expression cassette, a recombinant vector, a recombinant microorganism or a recombinant host cell containing the nucleic acid molecule.

2. The application according to claim 1, wherein the nucleic acid molecule is any one of m1) and m2):
m1) a DNA molecule with a nucleotide sequence of SEQ ID No. 2; or
m2) a DNA molecule having 75% or more identity to the nucleotide sequence in m1) and coding the PUB21 protein in claim 1.

3. Application of a substance that inhibits activity of a PUB21 protein or a substance that reduces a content of the PUB21 protein in any one of B1)-B5):
B1) improving Huanglongbing resistance of a plant;
B2) improving insect resistance of a plant;
B3) cultivating a Huanglongbing-resistant plant;
B4) cultivating an insect-resistant plant; or
B5) preventing and controlling Huanglongbing for a plant, wherein the PUB21 protein is any one of a1)-a4):
a1) a protein with an amino acid sequence of SEQ ID No. 1;
a2) a fused protein obtained by ligating a protein-tag at an N-terminus and/or a C-terminus of the amino acid sequence in a1);
a3) a protein with similar function obtained by subjecting the amino acid sequence in a1) to substitution and/or deletion and/or addition of one or several amino acid residues; or
a4) a protein having 75% or more identity to the amino acid sequence in a1) and having similar function.

4. The application according to claim 1, wherein the insect resistance is resistance to Diaphorina citri Kuwayama.

5. The application according to claim 1, wherein the plant is any one of P1)-P5):
P1) monocotyledonous plants or dicotyledonous plants;
P2) rutales plants;
P3) rutaceae plants;
P4) citrus plants; or
P5) citrus.

6. A PUB21 mutant protein, wherein the PUB21 mutant protein is a protein obtained by mutating cysteine at position 39 of the amino acid sequence of the PUB21 protein in claim 1 to alanine.

7. A biomaterial related to the PUB21 mutant protein according to claim 6, wherein the biomaterial is a nucleic acid molecule coding the PUB21 mutant protein, or an expression cassette, a recombinant vector, a recombinant microorganism or a recombinant host cell containing the nucleic acid molecule.

8. The biomaterial according to claim 7, wherein the nucleic acid molecule is any one of n1) and n2):
n1) a DNA molecule with a nucleotide sequence of SEQ ID No. 6; or
n2) a DNA molecule having 75% or more identity to the nucleotide sequence in n1) and coding the PUB21 mutant protein.

9. Application of the PUB21 mutant protein according to claim 6 or biomaterial related to the PUB21 mutant protein in any one of C1)-C5):
C1) improving Huanglongbing resistance of a plant;
C2) improving insect resistance of a plant;
C3) cultivating a Huanglongbing-resistant plant;
C4) cultivating an insect-resistant plant; or
C5) preventing and controlling Huanglongbing for a plant.

10. The application according to claim 9, wherein the insect resistance is resistance to Diaphorina citri Kuwayama.

11. The application according to claim 9, wherein the plant is any one of P1)-P5):
P1) monocotyledonous plants or dicotyledonous plants;
P2) rutales plants;
P3) rutaceae plants;
P4) citrus plants; or
P5) citrus.

12. A method for cultivating a Huanglongbing-resistant plant, wherein the method is any one of X1), or X2) or X3):
X1) reducing an expression and/or activity of the PUB21 protein in claim 1 in a recipient plant to obtain the Huanglongbing-resistant plant;
X2) editing a codon coding cysteine at position 39 of the PUB21 protein in claim 1 in the recipient plant to a codon coding other amino acids to obtain the Huanglongbing-resistant plant; or
X3) improving the expression and/or activity of the PUB21 protein wherein cysteine at position 39 of the amino acid sequence has been mutated to alanine in the recipient plant to obtain the Huanglongbing-resistant plant.

13. A method for cultivating an insect-resistant plant, wherein the method is any one of Y1), or Y2) or Y3):
Y1) reducing an expression and/or activity of the PUB21 protein in claim 1 in a recipient plant to obtain the insect-resistant plant;
Y2) editing a codon coding cysteine at position 39 of the PUB21 protein in claim 1 in the recipient plant to a codon coding other amino acids to obtain the insect-resistant plant; or
Y3) improving the expression and/or activity of the PUB21 protein wherein cysteine at position 39 of the amino acid sequence has been mutated to alanine in the recipient plant to obtain the insect-resistant plant.

14. The method according to claim 13, wherein the insect resistance is resistance to Diaphorina citri Kuwayama.

15. The method according to claim 12, wherein the other amino acids are alanine.

16. The method according to claim 12, wherein the plant is any one of P1)-P5):
P1) monocotyledonous plants or dicotyledonous plants;
P2) rutales plants;
P3) rutaceae plants;
P4) citrus plants; or
P5) citrus.

17. A Huanglongbing-resistant plant or an insect-resistant plant cultivated from the method according to claim 12.

18. A substance in which activity of PUB21 protein is inhibited or PUB21 protein level is reduced.

19. A substance in which MYC2 gene is overexpressed.

20. A method of preparing the substance of claim 18, wherein the substance can inhibit activity of PUB21 protein or reduce PUB21 level.
